(19) United States
(12) Patent Application Publication
(10) Pub. No.: US 2002/0086302 A1
(43) Pub. Date: Jul. 4, 2002

(54) Treatment methods using 17906 and uses therefor

(76) Inventors: David White, Braintree, ME (US); Kyle MacBeth, Boston, MA (US)

Correspondence Address: Carolyn A. Favorito, Morrison & Foerster LLP, Suite 500, 3811 Valley Centre Drive, San Diego, CA, 92130-2332, US

(21) Appl. No.: 09/860,193

(22) Filed: May 16, 2001

Related U.S. Application Data
(63) Continuation-in-part of application No. 09/571,689, filed on May 16, 2000.

(30) Foreign Application Priority Data
May 16, 2001 (US) PCT/US01/15835

(51) Int. Cl. C12Q 1/68; C07H 21/04; C12N 9/64; C12P 21/02; C12N 5/06

(52) U.S. Cl. 435/6; 435/226; 435/69.1; 435/325; 435/320.1; 536/23.2

(57) ABSTRACT

The present invention relates to methods and compositions for the diagnosis and treatment of bone associated or cellular proliferative or differentiative disease. Specifically, the present invention identifies 17906 genes which are differentially expressed in bone associated or cellular proliferative or differentiative disease states, relative to their expression in normal, or non-bone associated or non-cellular proliferative or differentiative disease states, and/or in response to manipulations relevant to bone associated or cellular proliferative or differentiative disease. The present invention describes methods for the diagnostic evaluation and prognosis of various bone associated or cellular proliferative or differentiative diseases, and for the identification of subjects exhibiting a predisposition to such conditions. The present invention provides methods for the diagnostic monitoring of patients undergoing clinical evaluation for the treatment of bone associated or cellular proliferative or differentiative disease, and for monitoring the efficacy of compounds in clinical trials. The present invention also provides methods for the identification and therapeutic use of compounds as treatments of bone associated or cellular proliferative or differentiative disease.

This application claims priority on U.S. application Ser. No. 09/571,689 filed May 16, 2000, which is relied on and incorporated herein by reference in its entirety.

### FIELD OF THE INVENTION

The invention relates to methods using previously characterized carboxypeptidase polypeptides and polynucleotides as targets for the diagnosis and treatment of bone-related and cellular proliferation and differentiation-related disorders.

### BACKGROUND OF THE INVENTION

Carboxypeptidases

Proteolytic enzymes are involved in many cellular processes. The carboxypeptidase family of enzymes catalyzes the cleavage of C-terminal amino acids of peptides and proteins, altering their biological activity. Lysosomal carboxypeptidase enzymes are highly concentrated in lysosomes, but may also be active extracellularly after their release from lysosomes in soluble form or bound to transmembrane or other membrane-associated proteins. Carboxypeptidases may cleave peptides in a sequence-specific manner. For example, prolylcarboxypeptidases cleave only peptides linked to proline residues (for example, des-Arg9-bradykinin, angiotensin II). There is also evidence that these enzymes are involved in terminating signal transduction by inactivating peptide ligands after receptor endocytosis.

In contrast to endoproteases which cleave internal peptide bonds of proteins and polypeptides, carboxypeptidases (CPs) catalyze the cleavage of only the C-terminal peptide bond, releasing one amino acid at a time. The two main groups of CPs include serine CPs and metallo-CPs, the serine CPs containing a signature trio of Ser, Asp, His in the active site. This trio is also contained in prolylendopeptidase serine proteases. Serine CPs include polycarboxypeptidase (PRCP) also referred to as angiotensinase C; and deamidase, also referred to as cathepsin A and lysosomal protective protein. See Skidgel et al. (1998) Immunological Reviews 161:129-141.

Metallo-CPs contain a signature glutamic acid as the primary catalytic residue and require zinc-binding for activity. Metallo-CPs can be grouped by substrate specificity into CPA and CPB types; the CPA type preferentially cleaving C-terminal hydrophobic residues, and the CPB type cleaving only peptides with C-terminal basic Arg or Lys residues. See R. A. Skidgel (1993) In: Hooper NM, ed. Zinc Metalloproteases in Health and Disease, London: Taylor & Francis, Ltd., p. 241-283.

CPM is a B type carboxypeptidase which is anchored on cell membranes via gylcosylphosphatidylinositol (GPI) association with its mildly hydrophobic stretch of 15 C-terminal amino acids. As in many other proteins sharing this anchoring mechanism, CPM is released from the membrane by bacterial phosphatidylinositol-specific phospholipase C. Human CPM is a glycoprotein of 426 amino acid residues with 43% identity to human intracellular secretory granular CP (CPE), 41% with the active 50 kDa subunit of human plasma CPN, and 15% with bovine pancreatic CPA or CPB. The active sites of these CPs contain conserved amino acid residues corresponding to the zinc binding residues His⁶⁶Glu⁶⁹ and His¹⁷³, substrate binding residues Arg¹³⁷ and Tyr²⁴², and the catalytic Glu²⁶⁴, as designated for CPM. Sequence homologies around these conserved residues is high, with an identity between CPs M, E and N of approximately 70-90%. See Tan et al. (1989) J. Biol. Chem. 264:13165-13170; Deddish et al. (1990) J. Biol. Chem. 265:15083-15089; R. A. Skidgel (1993) In: Hooper NM, ed. Zinc Metalloproteases in Health and Disease, London: Taylor & Francis, Ltd., p. 241-283. CPM has been mapped to the chromosomal location of chromosome 12q13-q15 which is associated with a variety of solid tumors.

The optimal pH range of CPM is in the neutral range of 6.5-7.5. As no endogenous inhibitors are known for CPM, the enzyme is considered to be constitutively active. Synthetic inhibitors including Arg analogs DL-2 mercaptomethyl-3-guanidinoethylthiopropanoic acid (MGTA) and guanidinoethylmercaptosuccinic acid (GEMSA) inhibit CPM. See R. A. Skidgel (1991) In: Conn PM, ed. Methods in Neurosciences: Peptide Technology Vol. 6, Orlando: Academic Press, p. 373-385; Plummer et al. (1981) Biochem. Biophys. Res. Comm. 98: 448-254.

As with other B type regulatory CPs, CPM cleaves only C-terminal Arg or Lys residues; however, CPM has a preference for the C-terminal Arg. The penultimate amino acid also affects the rate of hydrolysis. Naturally occurring peptide substrates of CPM include bradykinin, Arg⁶- and Lys⁶ enkephalins, dynorphin A¹⁻¹³ and epidermal growth factor (EGF). See Sidgel et al (1989) J. Biol. Chem. 264:2236-2241; McGwire et al (1995) J. Biol. Chem. 270:17154-17158.

CPM is primarily found on the plasma membrane, with highest levels found in lung and placenta. It is also present in kidney, blood vessels, intestine, brain and peripheral nerves. See R. A. Skidgel (1988) Trends Pharm. Sci. 9:299-304; Skidgel et al (1984) Biochem. Pharmacol. 33: 3471-3478; Skidgel et al (1991) FASEB J. 5:1578; Nagae et al (1992) J. Neurochem. 59:2201-2212; Nagae et al (1993) Am. J. Respir. Cell Mol. Biol. 9:221-229. Expression of CPM is responsive to differentiation of monocytes and lymphocytes. See de Saint-Vis et al (1995) Blood 86:1098-1105; Rehli et al (1995) J. Biol. Chem. 270:15644-15649.

CPM participates in the control of peptide hormone activity at the cell surface and degradation of extracellular proteins and peptides. It catalyzes the second step in prohormone processing and removes C-terminal Arg or Lys residues from peptides released from prohormones. CPM functions as a soluble enzyme after its release from the plasma membrane and may function in the plasma membrane form to control peptide receptor activities. CPM can regulate receptor specificity of kinins by cleaving the C-terminal ARG⁹, for example, from bradykinin. The intact bradykinin binds the B2 receptor. The cleaved bradykinin (des-ARG⁹-bradykinin). Des-ARG⁹-bradykinin also binds the B 1 receptors: stimulates IL-I and tumor necrosis factor release from macrophages. Regulation of the BI receptor is associated with injury or inflammation. CPM may also be involved with other inflammatory mediators, such as anaphylatoxin C5a which mediates histamine release. In addition, CPM may metabolize growth factors containing terminal Arg or Lys, such as EGF, EGF-like peptides, nerve growth factor (NGF) amphiregulin, hepatocyte growth factor, erythropoietin, and macrophage-stimulating protein. In the lung, varying levels of CPM are associated with pneumocystic or bacterial pneumonia or lung cancer, and in the placenta, CPM may protect the fetus from maternally derived peptides. See R. A. Skidgel (1992) J. Cardiovasc. Pharmacol. 20 (Suppl. 9):S4-S9; Bhoola et al. (1992) Pharmacol. Rev. 44:1-80; R. A. Skidgel (1993) In: Hooper NM, ed. Zinc Metalloproteases in Health and Disease, London: Taylor & Francis, Ltd., p. 241-283; Dragovic et al. (1995) Am. J. Respir. Crit. Care Med. 152:760-764; Nagae et al. (1992) J. Neurochem. 59:2201-2212; MacFadden et al. (1988) FASEB J. 2:1179 (Abstract).

Another B-type regulatory CP metalloprotein is CPD, a membrane-bound glycoprotein. Human CPD is a protein of 1,377 amino acids with 75% identity with duck GP180 and 90% identity with rat CPD. Human CPD contains two hydrophobic regions located at the C- and N-termini. A 55-60 residue cytoplasmic domain is highly conserved among duck, human and rat sequences and may be significant in intracellular sorting, protein-protein interactions or endocytosis. CPD contains three tandem CP homology domains numbered sequentially from the N- to the C-terminus, and thereby may contain more than one active site. See Tan et al. (1997) Biochem. J. 327:81-87; Skidgel et al. (1993) In: Robertson JLS, Nicholls MG, eds. The Renin Angiotensin System, Vol. 1, London: Gower Medical Publishing, p. 10.1-10.10. CPD is located on human chromosome 17, 17P, 11.1-17q, 11.2.

CPD is primarily found on intracellular membranes, mainly in the Golgi, with some CPD found on the plasma membrane. The tissue distribution of CPD is wide and includes most duck tissues and mammalian tissues as well, including brain, pituitary, placenta, pancreas, adrenal, kidney, lung, heart, spleen, intestine, ovary, and testes. See McGwire et al. (1997) Life Sci. 60:715-724; Song et al. (1995) J. Biol. Chem. 270:25007-25013; Xin et al. (1997) DNA Cell Biol. 16:897-909; Tan et al. (1997) Biochem. J. 327:81-87; Song et al. (1996) J. Biol. Chem. 271:28884-28889.

The function of CPD is speculated to include peptide and protein processing in the constitutive secretory pathway after endoprotease cleavage of precursor proteins. The enzyme has an acidic pH optimum. Mammalian CPD may act as a hepatitis B virus binding protein, similar to the duck CPD. See R. A. Skidgel (1998) Immunological Reviews 161:129-141.

Serine CPs include PRCP and deamidase. PRCP cloned from a human kidney library indicates a glycoprotein of 51 kDa³; and containing 496 amino acids, including a 30 residue signal peptide and a 15 residue propeptide. See Tan et al. (1993) J. Biol. Chem. 268:16631-16638. A serine repeat is found in the C-terminal half, similar to the serine repeat of a yeast CP encoded by the KEXI gene.

PRCP has an acidic pH optimum for synthetic peptide substrates, but retains activity at neutral ranges with longer naturally occurring peptides. PRCP cleaves peptides only if the penultimate residue is proline. The enzyme does not cleave Pro-Pro-COOH or (OH)-Pro-Pro-COOH bond. See Odya et al. (1978) J. Biol. Chem. 253:5927-5931. Substrates of PRCP include des-Arg⁹-bradykinin and angiotensin II.

PRCP may be involved in terminating signal transduction by inactivating peptide ligands after receptor endocytosis. PRCP is contained in lysosomes and released in response to stimulation. The enzyme is widely distributed and found in human placenta, lung, liver, and kidney.

Another serine CP, deamidase, is likely a 94 kDa homodimer of 52 kDa subunits. Human platelet deamidase is activated by cleavage of a 14 amino acid fragment from the C-terminus. The enzyme binds and maintains activity and stability of β-galactocidase and neuraminidase in lysosomes, a defect of which is associated with severe galactosialidosis. See Bonten et al. (1995) J. Biol. Chem. 270:26441-26445; Galjart et al. (1988) Cell 54:755-764; D'Azzo et al. (1982) Proc. Natl. Acad. Sci. 79:4535-4539. The gene for the human deamidase is mapped to chromosome 20 at q13.1.

Deamidase cleaves various peptides containing C-terminal or penultimate hydrophobic residues including substance P, angiotensin I, bradykinin, endothelin, and fMet-Leu-Phe. Like PRCP, deamidase is also found in lysosomes, and distributed in human placenta, lung, liver, and kidney. Like PRCP, deamidase is implicated in blocking part of the signal transduction pathway stimulated by peptides. Bradykinin, containing a C-terminal Arg⁹ and a penultimate hydrophobic amino acid Phe⁸, is cleaved by deamidase. Similarly, angiotensin, containing a C-terminal His and a penultimate Phe, is cleaved by deamidase. Accordingly, deamidase is implicated in termination of bradykinin activity on the B2 receptor to generate a BI receptor agonist. Deamidase may also have a role in chemotaxis and in metabolism of the anti-cancer growth factor antagonist. See Skidgel et al. (1998) Immunological Reviews 161:129-141; Jackman et al. (1990) J. Biol. Chem. 265:11265-11272; Jackman et al. (1995) Am. J. Respir. Cell Mol. Biol. 13:196-204; Hinek et al. (1996) Biol. Chem. 377:471-480; Jones et al. (1995) Peptides 16:777-783; Cummings et al. (1995) Biochem Pharmacol. 49:1709-1712.

Given the wide distribution and various physiological and pathological roles of carboxypeptidases, methods and compositions directed at regulating levels of these enzymes are useful for regulating peptide hormone activity, modulating metabolism of substance P, angiotensin I, angiotensin II, bradykinin, and endothelin, and regulation of signal transduction by inactivation of peptide ligands subsequent to receptor endocytosis.

Accordingly, carboxypeptidases are a major target for drug action and development.

The carboxypeptidase gene used in the methods of the invention (GenBank Accession AF095719) was purported to be involved in the histone hyperacetylation signaling pathway relating to prostate cancer differentiation. (Huang H. et al. Cancer Res. (1999) “Carboxypeptidase A3 (CPA3): a novel gene highly induced by histone deacetylase inhibitors during differentiation of prostate epithelial cancer cells” 15;59(12):2981-8). It was suggested that the CPA3 gene is involved in the histone hyperacetylation signaling pathway activated during NaBu-mediated differentiation of the androgen-independent prostate cancer cell line, PC-3 cells.

Bone Disorders

Human bone is subject to constant breakdown and re-synthesis in a complex process mediated by two cell types: osteoblasts, which produce new bone, and osteoclasts, which destroy bone. The activities of these two cell types are kept under control and in proper balance by a complex network of cytokines, growth factors and other cellular signals. It is understood that a number of known bone disorders may have their genesis in aberrant control of these cells. Likewise, a considerable amount of medical research has focussed on identifying the aspects of this control network which can be exploited to re-generate bone in patients with bone diseases.

Osteoporosis is one of several known degenerative bone disorders which can cause significant risk and hardship to those affected. It is generally defined as the gradual decrease in bone strength and density that occurs with advancing age, particularly among post-menopausal women. The clinical manifestations of osteoporosis include fractures of the vertebral bodies, the neck, and intertrochanteric regions of the femur, and the distal radius. Osteoporotic individuals may fracture any bone more easily than their nonosteoporotic counterparts. As many as many as 15-20 million individuals in the United States are afflicted with osteoporosis. About 1.3 million fractures attributable to osteoporosis occur annually in people age 45 and older. Among those who live to be age 90, 32 percent of women and 17 percent of men will suffer a hip fracture, mostly due to osteoporosis.

In addition to osteoporosis, there is a plethora of other conditions which are characterized by the need to enhance bone formation. Perhaps the most obvious is in the case of bone fractures, where it would be desirable to stimulate bone growth and to hasten and complete bone repair. Agents that enhance bone formation would also be useful in certain surgical procedures (e.g., facial reconstruction). Other conditions which result in a deficit or abnormal formation of bone include osteogenesis imperfecta (brittle bone disease), hypophosphatasia, Paget's disease, fibrous dysplasia, osteopetrosis, myeloma bone disease, and the depletion of calcium in bone which is related to primary hyperparathyroidism.

There are currently no pharmaceutical approaches to managing any of these conditions that is completely satisfactory. Bone deterioration associated with osteoporosis and other bone conditions may be treated with estrogens or bisphosphonates, which have known side effects, or with further invasive surgical procedures. Bone fractures are still treated exclusively using casts, braces, anchoring devices and other strictly mechanical means. More recently, surgical approaches to these types of injury utilize bovine or human cadaver bone which is chemically treated (to remove proteins) in order to prevent rejection. However, such bone implants, while mechanically important, are biologically dead (they do not contain bone-forming cells, growth factors, or other regulatory proteins). Thus, they do not greatly modulate the repair process. All of these concerns demonstrate a great need for new or novel forms of bone therapy.

### SUMMARY OF THE INVENTION

The present invention provides methods for the diagnosis and treatment of bone associated disease, including but not limited to, osteogenesis imperfecta (brittle bone disease), osteoporosis, Paget's disease (enlarged bones), fibrous dysplasia (uneven bone growth), hypophosphatasia, osteopetrosis, primary hyperthyroidism, or myeloma bone disease. The present invention is based, at least in part, on the discovery that the 17906 gene is down-regulated during osteoblast differentiation, and, thus, may be associated with a bone disorder.

In addition, the nucleic acid and protein molecules of the present invention are useful as modulating agents in regulating a variety of cellular processes, e.g., including cell proliferation, differentiation, growth and division. In particular, the nucleic acid and protein molecules of the present invention will be advantageous in the regulation of any cellular function involved in uncontrolled proliferation and differentiation, such as in cases of cancer. As such, the nucleic acid and protein molecules of the present invention provide methods for the diagnosis and treatment of cancer preferably, breast, ovarian, lung and colon tumors and metastases, and most preferably breast cancer. The present invention is based, at least in part, on the discovery that the 17906 gene is up-regulated in tumor cells, and, thus, may be associated with cancer.

In one aspect, the invention provides a method for identifying the presence of a nucleic acid molecule associated with a bone associated disorder or cellular proliferative or differentiative disorder in a sample by contacting a sample comprising nucleic acid molecules with a hybridization probe comprising at least 25 contiguous nucleotides of SEQ ID NO:1, and detecting the presence of a nucleic acid molecule associated with a bone associated disorder or cellular proliferative or differentiative disorder when the sample contains a nucleic acid molecule that hybridizes to the nucleic acid probe. In one embodiment, the hybridization probe is detectably labeled. In another embodiment the sample comprising nucleic acid molecules is subjected to agarose gel electrophoresis and southern blotting prior to contacting with the hybridization probe. In a further embodiment, the sample comprising nucleic acid molecules is subjected to agarose gel electrophoresis and northern blotting prior to contacting with the hybridization probe. In yet another embodiment, the detecting is by in situ hybridization. In other embodiments, the method is used to detect mRNA or genomic DNA in the sample.

The invention also provides a method for identifying a nucleic acid associated with a bone associated disorder or cellular proliferative or differentiative disorder or in a sample, by contacting a sample comprising nucleic acid molecules with a first and a second amplification primer, the first primer comprising at least 25 contiguous nucleotides of SEQ ID NO:1 and the second primer comprising at least 25 contiguous nucleotides from the complement of SEQ ID NO:1, incubating the sample under conditions that allow for nucleic acid amplification, and detecting the presence of a nucleic acid molecule associated with a bone associated disorder or cellular proliferative or differentiative disorder when the sample contains a nucleic acid molecule that is amplified. In one embodiment, the sample comprising nucleic acid molecules is subjected to agarose gel electrophoresis after the incubation step.

In addition, the invention provides a method for identifying a polypeptide associated with a bone associated disorder or cellular proliferative or differentiative disorder in a sample by contacting a sample comprising polypeptide molecules with a binding substance specific for a 17906 polypeptide, and detecting the presence of a polypeptide associated with a bone associated disorder or cellular proliferative or differentiative disorder when the sample contains a polypeptide molecule that binds to the binding substance. In one embodiment the binding substance is an antibody. In another embodiment, the binding substance is a 17906 ligand. In a further embodiment, the binding substance is detectably labeled.

In another aspect, the invention provides a method of identifying a subject at risk for a bone associated disorder or cellular proliferative or differentiative disorder by contacting a sample obtained from the subject comprising nucleic acid molecules with a hybridization probe comprising at least 25 contiguous nucleotides of SEQ ID NO:1, and detecting the presence of a nucleic acid molecule which identifies a subject a risk for a bone associated disorder or cellular proliferative or differentiative disorder when the sample contains a nucleic acid molecule that hybridizes to the nucleic acid probe.

In a further aspect, the invention provides a method for identifying a subject at risk for a bone associated disorder or cellular proliferative or differentiative disorder by contacting a sample obtained from a subject comprising nucleic acid molecules with a first and a second amplification primer, the first primer comprising at least 25 contiguous nucleotides of SEQ ID NO:1 and the second primer comprising at least 25 contiguous nucleotides from the complement of SEQ ID NO:1, incubating the sample under conditions that allow for nucleic acid amplification, and detecting a nucleic acid molecule which identifies a subject at risk for a bone associated disorder or cellular proliferative or differentiative disorder when the sample contains a nucleic acid molecule that is amplified.

In yet another aspect, the invention provides a method of identifying a subject at risk for a bone associated disorder or cellular proliferative or differentiative disorder by contacting a sample obtained from the subject comprising polypeptide molecules with a binding substance specific for a 17906 polypeptide, and identifying a subject at risk for a bone associated disorder or cellular proliferative or differentiative disorder by detecting the presence of a polypeptide molecule in the sample that binds to the binding substance.

In another aspect, the invention provides a method for identifying a compound capable of treating a bone associated disorder or cellular proliferative or differentiative disorder characterized by aberrant 17906 nucleic acid expression or 17906 protein activity by assaying the ability of the compound to modulate the expression of a 17906 nucleic acid or the activity of a 17906 protein. In one embodiment, the disorder is osteoporosis. In another embodiment, the disorder is cancer. In a further embodiment, the ability of the compound to modulate the activity of the 17906 protein is determined by detecting the induction of an intracellular second messenger.

In addition, the invention provides a method for treating a subject having a bone associated disorder or cellular proliferative or differentiative disorder characterized by aberrant 17906 protein activity or aberrant 17906 nucleic acid expression by administering to the subject a 17906 modulator. In one embodiment, the 17906 modulator is administered in a pharmaceutically acceptable formulation. In another embodiment the 17906 modulator is administered using a gene therapy vector. In a further embodiment, the 17906 modulator is a small molecule.

In one embodiment, a modulator is capable of modulating 17906 polypeptide activity. In another embodiment, the 17906 modulator is an anti-17906 antibody. In a further embodiment, the 17906 modulator is a 17906 polypeptide comprising the amino acid sequence of SEQ ID NO:2, or a fragment thereof. In yet another embodiment, the 17906 modulator is a 17906 polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4. In a further embodiment, the 17906 modulator is an isolated naturally occurring allelic variant of a polypeptide consisting of the amino acid sequence of SEQ ID NO:2, wherein the polypeptide is encoded by a nucleic acid molecule which hybridizes to a complement of a nucleic acid molecule consisting of SEQ ID NO:1 at 6× SSC at 45° C., followed by one or more washes in 0.2× SSC, 0.1% SDS at 50-65° C.

In one embodiment, the 17906 modulator is capable of modulating 17906 nucleic acid expression. In another embodiment, the 17906 modulator is an antisense 17906 nucleic acid molecule. In yet another embodiment, the 17906 modulator is a ribozyme. In a further embodiment, the 17906 modulator comprises the nucleotide sequence of SEQ ID NO:1, or a fragment thereof. In another embodiment, the 17906 modulator comprises a nucleic acid molecule encoding a polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4. In yet another embodiment, the 17906 modulator comprises a nucleic acid molecule encoding a naturally occurring allelic variant of a polypeptide comprising the amino acid sequence of SEQ ID NO:2, wherein the nucleic acid molecule which hybridizes to a complement of a nucleic acid molecule consisting of SEQ ID NO:1 at 6× SSC at 45° C., followed by one or more washes in 0.2× SSC, 0.1% SDS at 50-65° C.

In another aspect, the invention provides a method for identifying a compound capable of modulating a osteocyte activity by contacting a osteocyte with a test compound and assaying the ability of the test compound to modulate the expression of a 17906 nucleic acid or the activity of a 17906 protein. In certain embodiments, a compound that modulates the expression of a 17906 nucleic acid or the activity of a 17906 protein modulates osteocyte proliferation, migration, or the expression of cell surface adhesion molecules.

Furthermore, the invention provides a method for modulating a osteocyte activity comprising contacting a osteocyte with a 17906 modulator.

Other features and advantages of the invention will be apparent from the following detailed description and claims.

### BRIEF DESCRIPTION OF THE DRAWINGS

FIGS. 1a and b depicts the cDNA sequence and predicted amino acid sequence of human 17906 (GenBank Accession AF095719). The nucleotide sequence corresponds to nucleic acids 1 to 2795 of SEQ ID NO:1. The coding region corresponds to nucleic acids 8 to 1273 of SEQ ID NO:1. The amino acid sequence is identified as SEQ ID NO:2.

FIG. 2 is a graph depicting the results of real-time quantitative RT-PCR analysis of human 17906 expression in cells related to osteoblast differentiation.

FIG. 3 is a graph depicting the results of real-time quantitative RT-PCR analysis of human 17906 expression in various cell lines.

FIG. 4 is a graph depicting downregulation of human 17906 mRNA expression in osteoblasts treated with various compounds.

FIG. 5 is a graph depicting downregulation of human 17906 mRNA expression in primary osteoblasts (supplied from Clonectics) that are induced with β-phosphoglycerate to differentiate.

FIG. 6 is a breast model panel bar graph depicting the relative expression of 17906 mRNA relative to a no template control in a panel of cell lines, detected using real-time quantitative RT-PCR Taq Man analysis.

FIG. 7 is an oncology phase II bar graph depicting the expression of 17906 mRNA relative to a no template control showing an increased expression in 4/6 breast tumors in comparison to normal breast tissues, 5/5 ovarian tumors in comparison to normal ovarian tissues, 3/7 lung tumors in comparison to normal lung tissues, and 3/4 colon tumors and 2/2 colon metastases in comparison to normal colon tissues, which expression was detected using Taq Man analysis.

FIG. 8 is a panel bar graph depicting the relative expression of 17906 RNA relative to a no template control in a panel of human tissues or cells, including but not limited to artery, vein, aortic smooth muscle cells (SMC) (early), coronary SMC, shear and static human umbilical vein endothelial cells (HUVEC), heart, kidney, skeletal muscle, adipose, pancreas, osteoclasts, skin, spinal cord, brain cortex, brain hypothalamus, nerve dorsal root ganglia (DRS), glioblastoma, normal breast, breast tumor, normal ovary, ovary tumor, normal prostate and prostate tumor, epithelial, colon, liver, lung, fibroblasts, tonsil, bone marrow, activated PBMC, among others, detected using real-time quantitative RT-PCR Taq Man analysis. The graph indicates significant expression in prostate epithelial cells.

FIG. 9 depicts variable expression of 17906 in a xenograph panel.

### DETAILED DESCRIPTION OF THE INVENTION

The present invention provides methods and compositions for the diagnosis and treatment of bone associated disease, including but not limited to, osteogenesis imperfecta (brittle bone disease), osteoporosis, Paget's disease (enlarged bones), fibrous dysplasia (uneven bone growth), hypophosphatasia, osteopetrosis, primary hyperthyroidism, or myeloma bone disease. The present invention is based, at least in part, on the discovery that carboxypepsidase genes, referred to herein as “carboxypepsidase 17906” or “17906” nucleic acid and protein molecules, are downregulated during osteoblast differentiation, and, thus, may be associated with a bone disorder.

Aberrant expression and/or activity of 17906 molecules can mediate disorders associated with bone metabolism. “Bone metabolism” refers to direct or indirect effects in the formation or degeneration of bone structures, e.g., bone formation, bone resorption, etc., which can ultimately affect the concentrations in serum of calcium and phosphate. This term also includes activities mediated by 17906 molecules effects in bone cells, e.g. osteoclasts and osteoblasts, that can in turn result in bone formation and degeneration. For example, 17906 molecules can support different activities of bone resorbing osteoclasts such as the stimulation of differentiation of monocytes and mononuclear phagocytes into osteoclasts. Accordingly, 17906 molecules that modulate the production of bone cells can influence bone formation and degeneration, and thus can be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesisimperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

The present invention also provides methods and compositions for the diagnosis and treatment of cellular proliferative or differentiative associated disease, such as cancer, including but not limited to, breast, lung, ovarian, and colon cancer. The present invention is based, at least in part, on the discovery that carboxypepsidase genes, referred to herein as “carboxypepsidase 17906” or “17906” nucleic acid and protein molecules are up-regulated in some tumors and thus may be associated with a or cellular proliferative or differentiative disorder, such as cancer.

Thus, the 17906 molecules can act as novel diagnostic targets and therapeutic agents for controlling one or more cellular proliferative and/or differentiative disorders, such as cancer, and more specifically, breast, lung, brain and ovarian cancer. Examples of such disorders, e.g., phosphatase-associated or other 17906-associated disorders, include but are not limited to, cellular proliferative and/or differentiative disorders.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as nonpathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The 17906 molecules of the invention can be used to monitor, treat and/or diagnose a variety of proliferative disorders. Such disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, “differential expression” includes both quantitative as well as qualitative differences in the temporal and/or tissue expression pattern of a gene. Thus, a differentially expressed gene may have its expression activated or inactivated in normal versus bone associated or cellular proliferative or differentiative disease conditions. The degree to which expression differs in normal versus bone associated or cellular proliferative or differentiative disease or control versus experimental states need only be large enough to be visualized via standard characterization techniques, e.g. quantitative PCR, Northern analysis, or subtractive hybridization. The expression pattern of a differentially expressed gene may be used as part of a prognostic or diagnostic bone associated disease evaluation, or may be used in methods for identifying compounds useful for the treatment of bone associated or cellular proliferative or differentiative disease. In addition, a differentially expressed gene involved in bone associated or cellular proliferative or differentiative disease may represent a target gene such that modulation of the level of target gene expression or of target gene product activity may act to ameliorate a bone associated or cellular proliferative or differentiative disease condition. Compounds that modulate target gene expression or activity of the target gene product can be used in the treatment of bone or cellular proliferative or differentiative associated disease. Although the 17906 genes described herein may be differentially expressed with respect to bone or cellular proliferative or differentiative associated disease, and/or their products may interact with gene products important to bone or cellular proliferative or differentiative associated disease, the genes may also be involved in mechanisms important to additional bone or cellular proliferative or differentiative associated processes.

The 17906 molecules of the present invention may be involved in signal transduction and, thus, may that function to modulate cell proliferation, differentiation, and motility. Thus, the 17906 molecules of the present invention may play a role in cellular growth signaling mechanisms. As used herein, the term “cellular growth signaling mechanisms” includes signal transmission from cell receptors, e.g., G protein coupled receptors, which regulates 1) cell transversal through the cell cycle, 2) cell differentiation, 3) cell survival, and/or 4) cell migration and patterning.

Accordingly, the 17906 molecules of the present invention may be involved in cellular signal transduction pathways that modulate bone cell activity or cellular proliferation or differentiation. As used herein, a “bone cell activity”, “osteocyte activity”, or “bone cell function” includes cell proliferation differentiation, migration, and expression of cell surface adhesion molecules, as well as cellular process that contribute to the physiological role of bone cells (e.g., the regulation of calcium secretion). The 17906 molecules of the present invention also may play a role or function in the transduction of signals for cell proliferation, differentiation and apoptosis. In one embodiment, the 17906 molecules modulate the activity of one or more proteins involved in cellular growth or differentiation, e.g., breast, colon, lung or ovarian cell growth or differentiation.

Thus, the 17906 molecules, by participating in cellular growth signaling mechanisms, may modulate cell behavior and act as targets and therapeutic agents for controlling cellular proliferation and differentiation of bone cells or cells of the breast, colon, lung or ovaries.

The 17906 molecules of the present invention may also act as novel diagnostic targets and therapeutic agents for bone associated or cellular proliferative or differentiative diseases or disorders. As used herein, a “bone associated disease or disorder” includes a disease or disorder which affects bones. The term bone associated disorder includes a disorder affecting the normal function of the bones. For example, a bone associated disorder includes osteogenesis imperfecta (brittle bone disease), osteoporosis, Paget's disease (enlarged bones), fibrous dysplasia (uneven bone growth), hypophosphatasia, osteopetrosis, primary hyperthyroidism, or myeloma bone disease. bone associated disorders are described in, for example, Lamber et al. (2000) Pharmacotherapy 20:34-51; Eisman et al. (1999) Endocrine Reviews 20:788-804; Byers et al. (1992) Annual Rev. Med., 43:269-282; and at www.osteo.org.

As used herein, a “cellular proliferative or differentiative disorder” or a “cellular growth related disorder” includes a disorder, disease, or condition characterized by a deregulation, e.g., an upregulation or a downregulation, of cellular growth. Cellular growth deregulation may be due to a deregulation of cellular proliferation, cell cycle progression, cellular differentiation and/or cellular hypertrophy.

A bone associated disorder also includes a bone cell disorder. As used herein a “bone cell disorder” includes a disorder characterized by aberrant or unwanted bone cell activity, e.g., proliferation, migration, angiogenesis, or aberrant expression of cell surface adhesion molecules.

The present invention provides methods for identifying the presence of a 17906 nucleic acid or polypeptide molecule associated with a bone associated or cellular proliferative or differentiative disorder. In addition, the invention provides methods for identifying a subject at risk for a bone associated or cellular proliferative or differentiative disorder by detecting the presence of a 17906 nucleic acid or polypeptide molecule.

The invention also provides a method for identifying a compound capable of treating a bone associated or cellular proliferative or differentiative disorder characterized by aberrant 17906 nucleic acid expression or 17906 protein activity by assaying the ability of the compound to modulate the expression of a 17906 nucleic acid or the activity of a 17906 protein. Furthermore, the invention provides a method for treating a subject having a bone associated or cellular proliferative or differentiative disorder characterized by aberrant 17906 protein activity or aberrant 17906 nucleic acid expression by administering to the subject a 17906 modulator which is capable of modulating 17906 protein activity or 17906 nucleic acid expression.

Moreover, the invention provides a method for identifying a compound capable of modulating a bone or other cell activity by modulating the expression of a 17906 nucleic acid or the activity of a 17906 protein. The invention provides a method for modulating a bone or other cell activity comprising contacting a bone or other cell with a 17906 modulator.

Various aspects of the invention are described in further detail in the following subsections.

1. Screening Assays

The invention provides a method (also referred to herein as a “screening assay”) for identifying modulators, i.e., candidate or test compounds or agents (e.g., peptides, peptidomimetics, small molecules (organic or inorganic) or other drugs) which bind to 17906 proteins, have a stimulatory or inhibitory effect on, for example, 17906 expression or 17906 activity, or have a stimulatory or inhibitory effect on, for example, the expression or activity of a 17906 substrate.

These assays are designed to identify compounds that bind to a 17906 protein, bind to other cellular or extracellular proteins that interact with a 17906 protein, and interfere with the interaction of the 17906 protein with other cellular or extracellular proteins. For example, in the case of the 17906 protein, which is a transmembrane receptor-type protein, such techniques can identify ligands for such a receptor. A 17906 protein ligand can, for example, act as the basis for amelioration of bone associated or cellular proliferative or differentiative diseases, such as, for example, osteoporosis or cancer. Such compounds may include, but are not limited to peptides, antibodies, or small organic or inorganic compounds. Such compounds may also include other cellular proteins.

Compounds identified via assays such as those described herein may be useful, for example, for ameliorating bone associated or cellular proliferative or differentiative disease. In instances whereby a bone associated or cellular proliferative or differentiative disease condition results from an overall lower level of 17906 gene expression and/or 17906 protein in a cell or tissue, compounds that interact with the 17906 protein may include compounds which accentuate or amplify the activity of the bound 17906 protein. Such compounds would bring about an effective increase in the level of 17906 protein activity, thus ameliorating symptoms.

In other instances mutations within the 17906 gene may cause aberrant types or excessive amounts of 17906 proteins to be made which have a deleterious effect that leads to bone associated or cellular proliferative or differentiative disease. Similarly, physiological conditions may cause an excessive increase in 17906 gene expression leading to bone associated or cellular proliferative or differentiative disease. In such cases, compounds that bind to a 17906 protein may be identified that inhibit the activity of the 17906 protein. Assays for testing the effectiveness of compounds identified by techniques such as those described in this section are discussed herein.

In one embodiment, the invention provides assays for screening candidate or test compounds which are substrates of a 17906 protein or polypeptide or biologically active portion thereof. In another embodiment, the invention provides assays for screening candidate or test compounds which bind to or modulate the activity of a 17906 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 17906 protein or biologically active portion thereof is contacted with a test compound and the ability of the test compound to modulate 17906 activity is determined. Determining the ability of the test compound to modulate 17906 activity can be accomplished by monitoring, for example, intracellular calcium, IP3, CAMP, or diacylglycerol concentration, the phosphorylation profile of intracellular proteins, cell proliferation and/or migration, the expression of cell surface adhesion molecules, or the activity of a 17906-regulated transcription factor. The cell can be of mammalian origin, e.g., a bone, breast, ovarian, lung, or colon cell. In one embodiment, compounds that interact with a 17906 receptor domain can be screened for their ability to function as ligands, i.e., to bind to the 17906 receptor and modulate a signal transduction pathway. Identification of 17906 ligands, and measuring the activity of the ligand-receptor complex, leads to the identification of modulators (e.g., antagonists) of this interaction. Such modulators may be useful in the treatment of bone associated or cellular proliferative or differentiative disease.

The ability of the test compound to modulate 17906 binding to a substrate or to bind to 17906 can also be determined. Determining the ability of the test compound to modulate 17906 binding to a substrate can be accomplished, for example, by coupling the 17906 substrate with a radioisotope or enzymatic label such that binding of the 17906 substrate to 17906 can be determined by detecting the labeled 17906 substrate in a complex. 17906 could also be coupled with a radioisotope or enzymatic label to monitor the ability of a test compound to modulate 17906 binding to a 17906 substrate in a complex. Determining the ability of the test compound to bind 17906 can be accomplished, for example, by coupling the compound with a radioisotope or enzymatic label such that binding of the compound to 17906 can be determined by detecting the labeled 17906 compound in a complex. For example, compounds (e.g., 17906 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

The presence of 17906 in the serum of the transgenic and wild type animals can be determined by, for example, a carboxipeptidase assay. Briefly, 5 μl of serum of mice, for example, can be combined with 45 μl of 55 μM of an appropriate 17906 substrate including but not limited to e.g., angiotensin I, a kinin, or kinetensin, in 17906 buffer. Then, the rate of proteolytic degradation of the substrate can be measured by measuring the production of fluorescence (in fluorescence units) per second for 30 minutes at room temperature at a gain setting of 10. The average rate of fluorescence units per second (FU/sec) correlates directly with the amount of 17906 in the serum. As a control for the specificity of 17906, a standard carboxypeptidase assay can be performed (Holmquist and Riordan, Carboxypeptidase A, pp 44-60, Peptidase and their Inhibitors in Method of Enzymatic Analysis (1984)). Further, an additional carboxypeptidase assay can be performed in accordance with that described in Ostrowska, H. et al. (1998) Rocz Akad. Med. Bialymst., 43:39-55, which is incorporated herein by reference.

It is also within the scope of this invention to determine the ability of a compound (e.g., a 17906 ligand or substrate) to interact with 17906 without the labeling of any of the interactants. For example, a microphysiometer can be used to detect the interaction of a compound with 17906 without the labeling ofeither the compound or the 17906 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 17906.

In another embodiment, an assay is a cell-based assay comprising contacting a cell expressing a 17906 target molecule (e.g., a 17906 substrate) with a test compound and determining the ability of the test compound to modulate (e.g., stimulate or inhibit) the activity of the 17906 target molecule. Determining the ability of the test compound to modulate the activity of a 17906 target molecule can be accomplished, for example, by determining the ability of the 17906 protein to bind to or interact with the 17906 target molecule.

Determining the ability of the 17906 protein or a biologically active fragment thereof, to bind to or interact with a 17906 target molecule can be accomplished by one of the methods described above for determining direct binding. In a preferred embodiment, determining the ability of the 17906 protein to bind to or interact with a 17906 target molecule can be accomplished by determining the activity of the target molecule. For example, the activity of the target molecule can be determined by detecting induction of a cellular second messenger of the target (i.e., intracellular Ca²⁺, diacylglycerol, IP₃, CAMP), detecting catalytic/enzymatic activity of the target an appropriate substrate, detecting the induction of a reporter gene (comprising a targetresponsive regulatory element operatively linked to a nucleic acid encoding a detectable marker, e.g., luciferase), or detecting a target-regulated cellular response (e.g., cell proliferation or migration).

In yet another embodiment, an assay of the present invention is a cell-free assay in which a 17906 protein or biologically active portion thereof, is contacted with a test compound and the ability of the test compound to bind to the 17906 protein or biologically active portion thereof is determined. Preferred biologically active portions of the 17906 proteins to be used in assays of the present invention include fragments which participate in interactions with non-17906 molecules, e.g., fragments with high surface probability scores. Binding of the test compound to the 17906 protein can be determined either directly or indirectly as described above. In a preferred embodiment, the assay includes contacting the 17906 protein or biologically active portion thereof with a known compound which binds 17906 to form an assay mixture, contacting the assay mixture with a test compound, and determining the ability of the test compound to interact with a 17906 protein, wherein determining the ability of the test compound to interact with a 17906 protein comprises determining the ability of the test compound to preferentially bind to 17906 or biologically active portion thereof as compared to the known compound. Compounds that modulate the interaction of 17906 with a known target protein may be useful in regulating the activity of a 17906 protein, especially a mutant 17906 protein.

In another embodiment, the assay is a cell-free assay in which a 17906 protein or biologically active portion thereof is contacted with a test compound and the ability of the test compound to modulate (e.g., stimulate or inhibit) the activity of the 17906 protein or biologically active portion thereof is determined. Determining the ability of the test compound to modulate the activity of a 17906 protein can be accomplished, for example, by determining the ability of the 17906 protein to bind to a 17906 target molecule by one of the methods described above for determining direct binding. Determining the ability of the 17906 protein to bind to a 17906 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 17906 protein can be accomplished by determining the ability of the 17906 protein to further modulate the activity of a downstream effector of a 17906 target molecule. For example, the activity of the effector molecule on an appropriate target can be determined or the binding of the effector to an appropriate target can be determined as previously described.

In yet another embodiment, the cell-free assay involves contacting a 17906 protein or biologically active portion thereof with a known compound which binds the 17906 protein to form an assay mixture, contacting the assay mixture with a test compound, and determining the ability of the test compound to interact with the 17906 protein, wherein determining the ability of the test compound to interact with the 17906 protein comprises determining the ability of the 17906 protein to preferentially bind to or modulate the activity of a 17906 target molecule.

In more than one embodiment of the above assay methods of the present invention, it may be desirable to immobilize either 17906 or its target molecule to facilitate separation of complexed from uncomplexed forms of one or both of the proteins, as well as to accommodate automation of the assay. Binding of a test compound to a 17906 protein, or interaction of a 17906 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/17906 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 17906 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 17906 binding or activity determined using standard techniques.

Other techniques for immobilizing proteins on matrices can also be used in the screening assays of the invention. For example, either a 17906 protein or a 17906 target molecule can be immobilized utilizing conjugation of biotin and streptavidin. Biotinylated 17906 protein or target molecules can be prepared from biotin-NHS (Nhydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 17906 protein or target molecules but which do not interfere with binding of the 17906 protein to its target molecule can be derivatized to the wells of the plate, and unbound target or 17906 protein trapped in the wells by antibody conjugation. Methods for detecting such complexes, in addition to those described above for the GST-immobilized complexes, include immunodetection of complexes using antibodies reactive with the 17906 protein or target molecule, as well as enzyme-linked assays which rely on detecting an enzymatic activity associated with the 17906 protein or target molecule.

In another embodiment, modulators of 17906 expression are identified in a method wherein a cell is contacted with a candidate compound and the expression of 17906 mRNA or protein in the cell is determined. The level of expression of 17906 mRNA or protein in the presence of the candidate compound is compared to the level of expression of 17906 mRNA or protein in the absence of the candidate compound. The candidate compound can then be identified as a modulator of 17906 expression based on this comparison. For example, when expression of 17906 mRNA or protein is greater (statistically significantly greater) in the presence of the candidate compound than in its absence, the candidate compound is identified as a stimulator of 17906 mRNA or protein expression. Alternatively, when expression of 17906 mRNA or protein is less (statistically significantly less) in the presence of the candidate compound than in its absence, the candidate compound is identified as an inhibitor of 17906 mRNA or protein expression. The level of 17906 mRNA or protein expression in the cells can be determined by methods described herein for detecting 17906 mRNA or protein.

In yet another aspect of the invention, the 17906 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 17906 (“17906-binding proteins” or “17906-bp”) and are involved in 17906 activity. Such 17906-binding proteins are also likely to be involved in the propagation of signals by the 17906 proteins or 17906 targets as, for example, downstream elements of a 17906-mediated signaling pathway. Alternatively, such 17906-binding proteins are likely to be 17906 inhibitors.

The two-hybrid system is based on the modular nature of most transcription factors, which consist of separable DNA-binding and activation domains. Briefly, the assay utilizes two different DNA constructs. In one construct, the gene that codes for a 17906 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 17906-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 17906 protein.

In another aspect, the invention pertains to a combination of two or more of the assays described herein. For example, a modulating agent can be identified using a cell-based or a cell free assay, and the ability of the agent to modulate the activity of a 17906 protein can be confirmed in vivo, e.g., in an animal such as an animal model for bone associated or cellular proliferative or differentiative disease, as described herein.

This invention further pertains to novel agents identified by the above-described screening assays. Accordingly, it is within the scope of this invention to further use an agent identified as described herein in an appropriate animal model. For example, an agent identified as described herein (e.g., a 17906 modulating agent, an antisense 17906 nucleic acid molecule, a 17906-specific antibody, or a 17906-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate bone associated or cellular proliferative or differentiative disease symptoms. Cell-based and animal model-based assays for the identification of compounds exhibiting such an ability to ameliorate bone associated or cellular proliferative or differentiative disease systems are described herein.

In one aspect, cell-based systems, as described herein, may be used to identify compounds which may act to ameliorate bone associated or cellular proliferative or differentiative disease symptoms. For example, such cell systems may be exposed to a compound, suspected of exhibiting an ability to ameliorate bone associated or cellular proliferative or differentiative disease symptoms, at a sufficient concentration and for a time sufficient to elicit such an amelioration of bone associated or cellular proliferative or differentiative disease symptoms in the exposed cells. After exposure, the cells are examined to determine whether one or more of the bone associated or cellular proliferative or differentiative disease cellular phenotypes has been altered to resemble a more normal or more wild type phenotype. Cellular phenotypes that are associated with bone associated or cellular proliferative or differentiative disease states include aberrant proliferation and migration, deposition of extracellular matrix components, and expression of growth factors, cytokines, and other inflammatory mediators.

In addition, animal-based bone associated or cellular proliferative or differentiative disease systems, such as those described herein, may be used to identify compounds capable of ameliorating bone associated or cellular proliferative or differentiative disease symptoms. Such animal models may be used as test substrates for the identification of drugs, pharmaceuticals, therapies, and interventions which may be effective in treating bone associated or cellular proliferative or differentiative disease. For example, animal models may be exposed to a compound, suspected of exhibiting an ability to ameliorate bone associated or cellular proliferative or differentiative disease symptoms, at a sufficient concentration and for a time sufficient to elicit such an amelioration of bone associated or cellular proliferative or differentiative disease symptoms in the exposed animals. The response of the animals to the exposure may be monitored by assessing the reversal of disorders associated with bone associated or cellular proliferative or differentiative disease, for example, by measuring bone loss and/or measuring bone loss before and after treatment or the rate of cellular proliferation or differentiation.

With regard to intervention, any treatments which reverse any aspect of bone associated or cellular proliferative or differentiative disease symptoms should be considered as candidates for human bone associated or cellular proliferative or differentiative disease therapeutic intervention. Dosages of test agents may be determined by deriving dose-response curves.

Additionally, gene expression patterns may be utilized to assess the ability of a compound to ameliorate bone associated or cellular proliferative or differentiative disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, atherosclerosis, ischemia/reperfusion, hypertension, restenosis, and arterial inflammation, including any of the control or experimental conditions described herein. Gene expression profiles may be generated, for example, by utilizing a differential display procedure, Northern analysis and/or real-time quantitative RT-PCR. In one embodiment, 17906 gene sequences may be used as probes and/or PCR primers for the generation and corroboration of such gene expression profiles.

Gene expression profiles may be characterized for known states, either bone associated or cellular proliferative or differentiative disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a bone associated or cellular proliferative or differentiative disease model system to more closely resemble the control system. Administration of a compound may, alternatively, cause the gene expression profile of a control system to begin to mimic a bone associated or cellular proliferative or differentiative disease state. Such a compound may, for example, be used in further characterizing the compound of interest, or may be used in the generation of additional animal models.

2. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 17906 protein and/or nucleic acid expression as well as 17906 activity, in the context of a biological sample (e.g., blood, serum, cells, tissue) to thereby determine whether an individual is afflicted with a disease or disorder, or is at risk of developing a bone associated or cellular proliferative or differentiative disorder, associated with aberrant or unwanted 17906 expression or activity. The invention also provides for prognostic (or predictive) assays for determining whether an individual is at risk of developing a disorder associated with 17906 protein, nucleic acid expression or activity. For example, mutations in a 17906 gene can be assayed in a biological sample. Such assays can be used for prognostic or predictive purpose to thereby prophylactically treat an individual prior to the onset of a disorder characterized by or associated with 17906 protein, nucleic acid expression or activity.

Another aspect of the invention pertains to monitoring the influence of agents (e.g., drugs, compounds) on the expression or activity of 17906 in clinical trials.

These and other agents are described in further detail in the following sections.

A. Diagnostic Assays

The present invention encompasses methods for diagnostic and prognostic evaluation of bone associated or cellular proliferative or differentiative disease conditions, and for the identification of subjects exhibiting a predisposition to such conditions.

An exemplary method for detecting the presence or absence of 17906 protein or nucleic acid in a biological sample involves obtaining a biological sample from a test subject and contacting the biological sample with a compound or an agent capable of detecting 17906 protein or nucleic acid (e.g., mRNA, or genomic DNA) that encodes 17906 protein such that the presence of 17906 protein or nucleic acid is detected in the biological sample. A preferred agent for detecting 17906 mRNA or genomic DNA is a labeled nucleic acid probe capable of hybridizing to 17906 mRNA or genomic DNA. The nucleic acid probe can be, for example, the 17906 nucleic acid set forth in SEQ ID NO:1, or a portion thereof, such as an oligonucleotide of at least 15, 20, 25, 30, 35, 40, 45, 50, 100, 250 or 500 nucleotides in length and sufficient to specifically hybridize under stringent conditions to 17906 mRNA or genomic DNA. Other suitable probes for use in the diagnostic assays of the invention are described herein.

A preferred agent for detecting 17906 protein is an antibody capable of binding to 17906 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect 17906 mRNA, protein, or genomic DNA in a biological sample in vitro as well as in vivo. For example, in vitro techniques for detection of 17906 mRNA include Northern hybridizations and in situ hybridizations. In vitro techniques for detection of 17906 protein include enzyme linked immunosorbent assays (ELISAs), Western blots, immunoprecipitations and immunofluorescence. In vitro techniques for detection of 17906 genomic DNA include Southern hybridizations. Furthermore, in vivo techniques for detection of 17906 protein include introducing into a subject a labeled anti-17906 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting 17906 protein, mRNA, or genomic DNA, such that the presence of 17906 protein, mRNA or genomic DNA is detected in the biological sample, and comparing the presence of 17906 protein, mRNA or genomic DNA in the control sample with the presence of 17906 protein, mRNA or genomic DNA in the test sample.

The invention also encompasses kits for detecting the presence of 17906 in a biological sample. For example, the kit can comprise a labeled compound or agent capable of detecting 17906 protein or mRNA in a biological sample; means for determining the amount of 17906 in the sample; and means for comparing the amount of 17906 in the sample with a standard. The compound or agent can be packaged in a suitable container. The kit can further comprise instructions for using the kit to detect 17906 protein or nucleic acid.

B. Prognostic Assays

The diagnostic methods described herein can furthermore be utilized to identify subjects having or at risk of developing a bone associated or cellular proliferative or differentiative disease or disorder associated with aberrant or unwanted 17906 expression or activity. As used herein, the term “aberrant” includes a 17906 expression or activity which deviates from the wild type 17906 expression or activity. Aberrant expression or activity includes increased or decreased expression or activity, as well as expression or activity which does not follow the wild type developmental pattern of expression or the subcellular pattern of expression. For example, aberrant 17906 expression or activity is intended to include the cases in which a mutation in the 17906 gene causes the 17906 gene to be under-expressed or over-expressed and situations in which such mutations result in a non-functional 17906 protein or a protein which does not function in a wild-type fashion, e.g., a protein which does not interact with a 17906 ligand or substrate, or one which interacts with a non-I 7906 ligand or substrate. As used herein, the term “unwanted” includes an unwanted phenomenon involved in a biological response such as cellular proliferation. For example, the term unwanted includes a 17906 expression pattern or a 17906 protein activity which is undesirable in a subject.

The assays described herein, such as the preceding diagnostic assays or the following assays, can be utilized to identify a subject having or at risk of developing a disorder associated with a misregulation in 17906 protein activity or nucleic acid expression, such as a bone associated or cellular proliferative or differentiative disorder. Alternatively, the prognostic assays can be utilized to identify a subject having or at risk for developing a bone associated or cellular proliferative or differentiative disorder associated with a misregulation in 17906 protein activity or nucleic acid expression. Thus, the present invention provides a method for identifying a disease or disorder associated with aberrant or unwanted 17906 expression or activity in which a test sample is obtained from a subject and 17906 protein or nucleic acid (e.g., mRNA or genomic DNA) is detected, wherein the presence of 17906 protein or nucleic acid is diagnostic for a subject having or at risk of developing a disease or disorder associated with aberrant or unwanted 17906 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 17906 expression or activity. For example, such methods can be used to determine whether a subject can be effectively treated with an agent for a bone associated or cellular proliferative or differentiative disorder. Thus, the present invention provides methods for determining whether a subject can be effectively treated with an agent for a bone associated or cellular proliferative or differentiative disorder associated with aberrant or unwanted 17906 expression or activity in which a test sample is obtained and 17906 protein or nucleic acid expression or activity is detected (e.g., wherein the abundance of 17906 protein or nucleic acid expression or activity is diagnostic for a subject that can be administered the agent to treat a disorder associated with aberrant or unwanted 17906 expression or activity).

The methods of the invention can also be used to detect genetic alterations in a 17906 gene, thereby determining if a subject with the altered gene is at risk for a disorder characterized by misregulation in 17906 protein activity or nucleic acid expression, such as a proliferative disorder. In preferred embodiments, the methods include detecting, in a sample of cells from the subject, the presence or absence of a genetic alteration characterized by at least one of an alteration affecting the integrity of a gene encoding a 17906-protein, or the mis-expression of the 17906 gene. For example, such genetic alterations can be detected by ascertaining the existence of at least one of 1) a deletion of one or more nucleotides from a 17906 gene; 2) an addition of one or more nucleotides to a 17906 gene; 3) a substitution of one or more nucleotides of a 17906 gene, 4) a chromosomal rearrangement of a 17906 gene; 5) an alteration in the level of a messenger RNA transcript of a 17906 gene, 6) aberrant modification of a 17906 gene, such as of the methylation pattern of the genomic DNA, 7) the presence of a non-wild type splicing pattern of a messenger RNA transcript of a 17906 gene, 8) a non-wild type level of a 17906-protein, 9) allelic loss of a 17906 gene, and 10) inappropriate post-translational modification of a 17906-protein. As described herein, there are a large number of assays known in the art which can be used for detecting alterations in a 17906 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the 17906-gene (see Abravaya et al. (1995) Nucleic Acids Res 0.23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a 17906 gene under conditions such that hybridization and amplification of the 17906-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Other amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 17906 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 17906 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in 17906 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 17906 gene and detect mutations by comparing the sequence of the sample 17906 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 17906 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type 17906 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 17906 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 17906 sequence, e.g., a wild-type 17906 sequence, is hybridized to a cDNA or other DNA product from a test cell(s). The duplex is treated with a DNA mismatch repair enzyme, and the cleavage products, if any, can be detected from electrophoresis protocols or the like (described in, for example, U.S. Pat. No. 5,459,039).

In other embodiments, alterations in electrophoretic mobility will be used to identify mutations in 17906 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control 17906 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of highmelting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g. in clinical settings to diagnose patients exhibiting symptoms or family history of a disease or illness involving a 17906 gene.

Furthermore, any cell type or tissue in which 17906 is expressed may be utilized in the prognostic assays described herein.

C. Monitoring of Effects During Clinical Trials

The present invention provides methods for evaluating the efficacy of drugs and monitoring the progress of patients involved in clinical trials for the treatment of bone associated or cellular proliferative or differentiative disease.

Monitoring the influence of agents (e.g., drugs) on the expression or activity of a 17906 protein (e.g., the modulation of cell proliferation and/or migration) can be applied not only in basic drug screening, but also in clinical trials. For example, the effectiveness of an agent determined by a screening assay as described herein to increase 17906 gene expression, protein levels, or upregulate 17906 activity, can be monitored in clinical trials of subjects exhibiting decreased 17906 gene expression, protein levels, or downregulated 17906 activity. Alternatively, the effectiveness of an agent determined by a screening assay to decrease 17906 gene expression, protein levels, or downregulate 17906 activity, can be monitored in clinical trials of subjects exhibiting increased 17906 gene expression, protein levels, or upregulated 17906 activity. In such clinical trials, the expression or activity of a 17906 gene, and preferably, other genes that have been implicated in, for example, a 17906-associated disorder can be used as a “read out” or markers of the phenotype a particular cell, e.g., a bone, breast, lung, ovary or colon cell. In addition, the expression of a 17906 gene, or the level of 17906 protein activity may be used as a read out of a particular drug or agent's effect on a bone associated or cellular proliferative or differentiative disease state.

For example, and not by way of limitation, genes, including 17906, that are modulated in cells by treatment with an agent (e.g., compound, drug or small molecule) which modulates 17906 activity (e.g., identified in a screening assay as described herein) can be identified. Thus, to study the effect of agents on 17906-associated disorders (e.g., bone associated or cellular proliferative or differentiative disorders characterized by deregulated bone, breast, lung, ovary or colon cell activity), for example, in a clinical trial, cells can be isolated and RNA prepared and analyzed for the levels of expression of 17906 and other genes implicated in the 17906-associated disorder, respectively. The levels of gene expression (e.g., a gene expression pattern) can be quantified by northern blot analysis or real-time quantitative RT-PCR, as described herein, or alternatively by measuring the amount of protein produced, by one of the methods as described herein, or by measuring the levels of activity of 17906 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a 17906 protein, mRNA, or genomic DNA in the preadministration sample; (iii) obtaining one or more post-administration samples from the subject; (iv) detecting the level of expression or activity of the 17906 protein, mRNA, or genomic DNA in the post-administration samples; (v) comparing the level of expression or activity of the 17906 protein, mRNA, or genomic DNA in the preadministration sample with the 17906 protein, mRNA, or genomic DNA in the post administration sample or samples; and (vi) altering the administration of the agent to the subject accordingly. For example, increased administration of the agent may be desirable to increase the expression or activity of 17906 to higher levels than detected, i.e., to increase the effectiveness of the agent. Alternatively, decreased administration of the agent may be desirable to decrease expression or activity of 17906 to lower levels than detected, i.e. to decrease the effectiveness of the agent. According to such an embodiment, 17906 expression or activity may be used as an indicator of the effectiveness of an agent, even in the absence of an observable phenotypic response.

3. Use of 17906 Molecules as Surrogate Markers

The 17906 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 17906 molecules of the invention may be detected, and may be correlated with one or more biological states in vivo. For example, the 17906 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 17906 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a 17906 marker) transcription or expression, the amplified marker may be in a quantity which is more readily detectable than the drug itself. Also, the marker may be more easily detected due to the nature of the marker itself; for example, using the methods described herein, anti-17906 antibodies may be employed in an immune-based detection system for a 17906 protein marker, or 17906-specific radiolabeled probes may be used to detect a 17906 mRNA marker. Furthennore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Phann. 56 Suppl. 3: S16-S20.

The 17906 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 17906 protein or RNA) for specific tumor markers in a subject, a drug or course of treatment may be selected that is optimized for the treatment of the specific tumor likely to be present in the subject. Similarly, the presence or absence of a specific sequence mutation in 17906 DNA may correlate 17906 drug response. The use of pharmacogenomic markers therefore permits the application of the most appropriate treatment for each subject without having to administer the therapy.

4. Methods of Treatment:

The present invention provides for both prophylactic and therapeutic methods of treating a subject at risk of (or susceptible to) a disorder or having a disorder associated with aberrant or unwanted 17906 expression or activity, e.g. a bone associated or cellular proliferative or differentiative disorder. With regards to both prophylactic and therapeutic methods of treatment, such treatments may be specifically tailored or modified, based on knowledge obtained from the field of pharmacogenomics. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 17906 molecules of the present invention or 17906 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a bone associated or cellular proliferative or differentiative disease or condition associated with an aberrant or unwanted 17906 expression or activity, by administering to the subject a 17906 or an agent which modulates 17906 expression or at least one 17906 activity. Subjects at risk for a bone associated or cellular proliferative or differentiative disease which is caused or contributed to by aberrant or unwanted 17906 expression or activity can be identified by, for example, any or a combination of diagnostic or prognostic assays as described herein. Administration of a prophylactic agent can occur prior to the manifestation of symptoms characteristic of the 17906 aberrancy, such that a disease or disorder is prevented or, alternatively, delayed in its progression. Depending on the type of 17906 aberrancy, for example, a 17906, 17906 agonist or 17906 antagonist agent can be used for treating the subject. The appropriate agent can be determined based on screening assays described herein.

B. Therapeutic Methods

Described herein are methods and compositions whereby bone associated or cellular proliferative or differentiative disease symptoms may be ameliorated. Certain bone associated or cellular proliferative or differentiative diseases are brought about, at least in part, by an excessive level of a gene product, or by the presence of a gene product exhibiting an abnormal or excessive activity. As such, the reduction in the level and/or activity of such gene products would bring about the amelioration of bone associated or cellular proliferative or differentiative disease symptoms. Techniques for the reduction of gene expression levels or the activity of a protein are discussed below.

Alternatively, certain other bone associated or cellular proliferative or differentiative diseases are brought about, at least in part, by the absence or reduction of the level of gene expression, or a reduction in the level of a protein's activity. As such, an increase in the level of gene expression and/or the activity of such proteins would bring about the amelioration of bone associated or cellular proliferative or differentiative disease symptoms.

In some cases, the up-regulation of a gene in a disease state reflects a protective role for that gene product in responding to the disease condition. Enhancement of such a gene's expression, or the activity of the gene product, will reinforce the protective effect it exerts. Some bone associated or cellular proliferative or differentiative disease states may result from an abnormally low level of activity of such a protective gene. In these cases also, an increase in the level of gene expression and/or the activity of such gene products would bring about the amelioration of bone associated or cellular proliferative or differentiative disease symptoms. Techniques for increasing target gene expression levels or target gene product activity levels are discussed herein.

Accordingly, another aspect of the invention pertains to methods of modulating 17906 expression or activity for therapeutic purposes. Accordingly, in an exemplary embodiment, the modulatory method of the invention involves contacting a cell with a 17906 or agent that modulates one or more of the activities of 17906 protein activity associated with the cell (e.g., a bone, breast, lung, colon or ovarian cell). An agent that modulates 17906 protein activity can be an agent as described herein, such as a nucleic acid or a protein, a naturally-occurring target molecule of a 17906 protein (e.g., a 17906 ligand or substrate), a 17906 antibody, a 17906 agonist or antagonist, a peptidomimetic of a 17906 agonist or antagonist, or other small molecule. In one embodiment, the agent stimulates one or more 17906 activities. Examples of such stimulatory agents include active 17906 protein and a nucleic acid molecule encoding 17906 that has been introduced into the cell. In another embodiment, the agent inhibits one or more 17906 activities. Examples of such inhibitory agents include antisense 17906 nucleic acid molecules, anti-17906 antibodies, and 17906 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 17906 protein or nucleic acid molecule. In one embodiment, the method involves administering an agent (e.g., an agent identified by a screening assay described herein), or combination of agents that modulates (e.g., upregulates or downregulates) 17906 expression or activity. In another embodiment, the method involves administering a 17906 protein or nucleic acid molecule as therapy to compensate for reduced, aberrant, or unwanted 17906 expression or activity.

Stimulation of 17906 activity is desirable in situations in which 17906 is abnormally downregulated and/or in which increased 17906 activity is likely to have a beneficial effect. Likewise, inhibition of 17906 activity is desirable in situations in which 17906 is abnormally upregulated and/or in which decreased 17906 activity is likely to have a beneficial effect.

Preferably, the 17906 molecules can act as novel diagnostic targets and therapeutic agents for controlling a bone disorder or one or more cellular proliferative and/or differentiative disorders, such as cancer, and more specifically, breast, lung, brain and ovarian cancer. In addition, the 17906 molecules can act as novel diagnostic targets and therapeutic agents for controlling other disorders, e.g., phosphatase-associated or other 17906-associated disorders, that include but are not limited to, cellular proliferative and/or differentiative disorders, disorders associated with bone metabolism, immune e.g., inflammatory, disorders, cardiovascular disorders, including endothelial cell disorders, liver disorders, viral diseases, pain or metabolic disorders.

The 17906 nucleic acid and protein of the invention can be used to treat and/or diagnose a variety of immune, e.g., inflammatory, (e.g. respiratory inflammatory) disorders. Examples of immune disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, inflammatory bowel disease, e.g. Crohn's disease and ulcerative colitis, aphthous ulcer, iritis, conjunctivitis, keratoconjunctivitis, asthma, allergic asthma, chronic obstructive pulmonary disease, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Examples of disorders involving the heart or “cardiovascular disorder” include, but are not limited to, a disease, disorder, or state involving the cardiovascular system, e.g., the heart, the blood vessels, and/or the blood. A cardiovascular disorder can be caused by an imbalance in arterial pressure, a malfinction of the heart, or an occlusion of a blood vessel, e.g., by a thrombus. Examples of cardiovascular disorders include but are not limited to, hypertension, atherosclerosis, coronary artery spasm, coronary artery disease, arrhythmias, heart failure, including but not limited to, cardiac hypertrophy, leftsided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, disorders involving cardiac transplantation, and congestive heart failure.

A cardiovascular disease or disorder also includes an endothelial cell disorder. As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, Al-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 17906 molecules can play an important role in the etiology of certain viral diseases, including but not limited to Hepatitis B, Hepatitis C and Herpes Simplex Virus (HSV). Modulators of 17906 activity could be used to control viral diseases. The modulators can be used in the treatment and/or diagnosis of viral infected tissue or virus-associated tissue fibrosis, especially liver and liver fibrosis. Also, 17906 modulators can be used in the treatment and/or diagnosis of virus-associated carcinoma, especially hepatocellular cancer.

Additionally, 17906 can play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

(i) Methods for Inhibiting Target Gene Expression, Synthesis, or Activity

As discussed above, genes involved in bone associated or cellular proliferative or differentiative disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate bone associated or cellular proliferative or differentiative disease symptoms. Such molecules may include, but are not limited to, small organic molecules, peptides, antibodies, and the like.

For example, compounds can be administered that compete with endogenous ligand for the 17906 protein. The resulting reduction in the amount of ligand-bound 17906 protein will modulate bone, breast, lung, colon or ovarian cell physiology. Compounds that can be particularly useful for this purpose include, for example, soluble proteins or peptides, such as peptides comprising one or more of the extracellular domains, or portions and/or analogs thereof, of the 17906 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 17906 receptor site, but do not activate the protein, (e.g., receptor-ligand antagonists) can be effective in inhibiting 17906 protein activity.

Further, antisense and ribozyme molecules which inhibit expression of the 17906 gene may also be used in accordance with the invention to inhibit aberrant 17906 gene activity. Still further, triple helix molecules may be utilized in inhibiting aberrant 17906 gene activity.

The antisense nucleic acid molecules of the invention are typically administered to a subject or generated in situ such that they hybridize with or bind to cellular mRNA and/or genomic DNA encoding a 17906 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 17906 mRNA transcripts to thereby inhibit translation of 17906 mRNA. A ribozyme having specificity for a 17906-encoding nucleic acid can be designed based upon the nucleotide sequence of a 17906 cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a 17906-encoding mRNA (see, for example, Cech et al. U.S. Pat. No. 4,987,071; and Cech et al. U.S. Pat. No. 5,116,742). Alternatively, 17906 mRNA can be used to select a catalytic RNA having a specific ribonuclease activity from a pool of RNA molecules (see, for example, Bartel, D. and Szostak, J. W. (1993) Science 261:1411-1418).

17906 gene expression can also be inhibited by targeting nucleotide sequences complementary to the regulatory region of the 17906 (e.g., the 17906 promoter and/or enhancers) to form triple helical structures that prevent transcription of the 17906 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 17906 protein and interfere with its activity may also be used to modulate or inhibit 17906 protein function. Such antibodies may be generated using standard techniques described herein, against the 17906 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 17906 protein. Antibodies that are specific for one or more extracellular domains of the 17906 protein, for example, and that interfere with its activity, are particularly useful in treating bone associated or cellular proliferative or differentiative disease. Such antibodies are especially efficient because they can access the target domains directly from the bloodstream. Any of the administration techniques described below which are appropriate for peptide administration may be utilized to effectively administer inhibitory target gene antibodies to their site of action.

(ii) Methods for Restoring Enhancing or Inhibiting Target Gene Activity

Described in this section are methods whereby the level 17906 activity may be modulated to levels wherein bone associated or cellular proliferative or differentiative disease symptoms are ameliorated. The level of 17906 activity may be modulated, for example, by either modulating the level of 17906 gene expression or by modulating the level of active 17906 protein which is present.

Specifically, 17906 is down-regulated in osteoblast differentiation, thus 17906 may be used to modulate osteoblast activity, either by increasing 17906 activity and promoting bone cell proliferation or inhibiting 17906 activity and promoting bone cell differentiation, for example. Modulation to further decrease differentiation and to allow bone cells to proliferate is useful for bone regeneration and thus useful for treating diseases such as osteoporosis. Modulation to increase differentiation and reduce proliferation is useful for reducing bone cell growth and thus is useful for treating diseases such as myeloma bone disease.

17906 is up-regulated in tumors, thus 17906 may be used to modulate cellular activity by inhibiting 17906 activity and inhibiting tumor cell differentiation, for example.

Genes that cause bone associated or cellular proliferative or differentiative disease may be underexpressed within bone associated or cellular proliferative or differentiative disease situations. Bone associated or cellular proliferative or differentiative disease symptoms may also develop due to the decrease of activity of the protein products of such genes. Such down-regulation of gene expression or decrease of protein activity might have a causative or exacerbating effect on the disease state.

In some cases, genes that are down-regulated in the disease state might be exerting a protective effect. A variety of techniques may be used to decrease the expression, synthesis, or activity of 17906 genes and/or proteins that exert a causatory effect on bone associated or cellular proliferative or differentiative disease conditions.

In contrast, an inhibitor of a 17906 protein, at a level sufficient to ameliorate bone associated or cellular proliferative or differentiative disease symptoms may be administered to a patient exhibiting such symptoms. Any of the techniques discussed below may be used for such administration. One of skill in the art will readily know how to determine the concentration of effective, non-toxic doses of an inhibitor of the 17906 protein, utilizing techniques such as those described below.

Additionally, antisense 17906 DNA sequences may be directly administered to a patient exhibiting bone associated or cellular proliferative or differentiative disease symptoms, at a concentration sufficient to reduce the level of 17906 protein such that bone associated or cellular proliferative or differentiative disease symptoms are ameliorated. Any of the techniques discussed below, which achieve intracellular administration of compounds, such as, for example, liposome administration, may be used for the administration of such antisense DNA molecules. The DNA molecules may be produced, for example, by recombinant techniques such as those described herein.

Further, subjects may be treated by gene replacement therapy. One or more copies of an antagonist of the 17906 molecule, e.g., a portion of the 17906 gene, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 17906 gene sequences into human cells.

Cells, preferably, autologous cells, containing 17906 antagonist expressing gene sequences may then be introduced or reintroduced into the subject at positions which allow for the amelioration of bone associated or cellular proliferative or differentiative disease symptoms. Such cell replacement techniques may be preferred, for example, when the gene product is a secreted, extracellular gene product.

C. Pharmacogenomics

The 17906 molecules of the present invention, as well as agents, or modulators which have a stimulatory or inhibitory effect on 17906 activity (e.g., 17906 gene expression) as identified by a screening assay described herein can be administered to individuals to treat (prophylactically or therapeutically) 17906-associated disorders (e.g., bone associated or cellular proliferative or differentiative disorders) associated with aberrant or unwanted 17906 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a 17906 molecule or a 17906 modulator as well as tailoring the dosage and/or therapeutic regimen of treatment with a 17906 molecule or 17906 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(1011): 983-985 and Linder, M. W. et a. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the 3 human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a 17906 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a 17906 molecule or 17906 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a 17906 molecule or 17906 modulator, such as a modulator identified by one of the exemplary screening assays described herein.

5. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

A. Chromosome Mapping

Once the sequence (or a portion of the sequence) of a gene has been isolated, this sequence can be used to map the location of the gene on a chromosome. This process is called chromosome mapping. Accordingly, portions or fragments of the 17906 nucleotide sequences, described herein, can be used to map the location of the 17906 genes on a chromosome. The mapping of the 17906 sequences to chromosomes is an important first step in correlating these sequences with genes associated with disease. The 17906 gene has been mapped to human chromosome position 15q14-15.

Briefly, 17906 genes can be mapped to chromosomes by preparing PCR primers (preferably 15-25 bp in length) from the 17906 nucleotide sequences. Computer analysis of the 17906 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the 17906 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the 17906 nucleotide sequences to design oligonucleotide primers, sublocalization can be achieved with panels of fragments from specific chromosomes. Other mapping strategies which can similarly be used to map a 17906 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the 17906 gene, can be deterrnined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

B. Tissue Typing

The 17906 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the 17906 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The 17906 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of 17906 gene sequences can comfortably provide positive individual identification with a panel of perhaps 10 to 1,000 primers which each yield a noncoding amplified sequence of 100 bases. If predicted coding sequences, such as those in SEQ ID NO:1 are used, a more appropriate number of primers for positive individual identification would be 500-2,000.

If a panel of reagents from 17906 nucleotide sequences described herein is used to generate a unique identification database for an individual, those same reagents can later be used to identify tissue from that individual. Using the unique identification database, positive identification of the individual, living or dead, can be made from extremely small tissue samples.

C. Use of Partial 17906 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g. hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of 17906 gene sequences are particularly appropriate for this use as greater numbers of polymorphisms occur in the noncoding regions, making it easier to differentiate individuals using this technique. Examples of polynucleotide reagents include the 17906 nucleotide sequences or portions thereof, e.g., fragments derived from the noncoding regions having a length of at least 20 bases, preferably at least 30 bases.

The 17906 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such 17906 probes can be used to identify tissue by species and/or by organ type.

In a similar fashion, these reagents, e.g., 17906 primers or probes can be used to screen tissue culture for contamination (i.e. screen for the presence of a mixture of different types of cells in a culture).

6. Recombinant Expression Vectors and Host Cells

The methods of the invention include the use of vectors, preferably expression vectors, containing a nucleic acid encoding a 17906 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the methods of the invention may include other forms of expression vectors, such as viral vectors (e.g., replication defective retroviruses, adenoviruses and adenoassociated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g. tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 17906 proteins, mutant forms of 17906 proteins, fusion proteins, and the like).

The recombinant expression vectors of the invention can be designed for expression of 17906 proteins in prokaryotic or eukaryotic cells, e.g., for use in the cell-based assays of the invention. For example, 17906 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 17906 activity assays, (e.g., direct assays or competitive assays described in detail below), or to generate antibodies specific for 17906 proteins, for example. In a preferred embodiment, a 17906 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the 17906 expression vector is a yeast expression vector. Examples of vectors for expression in yeast S. cerevisiae include pYepSecl (Baldari, et al., (1987) EMBOJ 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, 17906 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g. the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the a-fetoprotein promoter (Campes and Tilghman (1989) Genes Dev. 3:537-546).

The expression characteristics of an endogenous 17906 gene within a cell line or microorganism may be modified by inserting a heterologous DNA regulatory element into the genome of a stable cell line or cloned microorganism such that the inserted regulatory element is operatively linked with the endogenous 17906 gene. For example, an endogenous 17906 gene which is normally “transcriptionally silent”, i.e., a 17906 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous 17906 gene may be activated by insertion of a promiscuous regulatory element that works across cell types.

A heterologous regulatory element may be inserted into a stable cell line or cloned microorganism, such that it is operatively linked with an endogenous 17906 gene, using techniques, such as targeted homologous recombination, which are well known to those of skill in the art, and described, e.g., in Chappel, U.S. Pat. No. 5,272,071; PCT publication No. WO 91/06667, published May 16, 1991.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 17906 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a 17906 nucleic acid molecule of the invention is introduced, e.g., a 17906 nucleic acid molecule within a recombinant expression vector or a 17906 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 17906 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a 17906 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a 17906 protein. Accordingly, the invention further provides methods for producing a 17906 protein using the host cells of the invention. In one embodiment, the method comprises culturing the host cell of the invention (into which a recombinant expression vector encoding a 17906 protein has been introduced) in a suitable medium such that a 17906 protein is produced. In another embodiment, the method further comprises isolating a 17906 protein from the medium or the host cell.

7. Cell- and Animal-Based Model Systems

Described herein are cell- and animal-based systems which act as models for bone associated or cellular proliferative or differentiative disease. These systems may be used in a variety of applications. For example, the cell- and animal-based model systems may be used to further characterize differentially expressed genes associated with bone associated or cellular proliferative or differentiative disease, e.g., 17906. In addition, animal- and cell-based assays may be used as part of screening strategies designed to identify compounds which are capable of ameliorating bone associated or cellular proliferative or differentiative disease symptoms, as described, below. Thus, the animal- and cell-based models may be used to identify drugs, pharmaceuticals, therapies and interventions which may be effective in treating bone associated or cellular proliferative or differentiative disease. Furthermore, such animal models may be used to determine the LD50 and the ED50 in animal subjects, and such data can be used to determine the in vivo efficacy of potential bone associated or cellular proliferative or differentiative disease treatments.

A. Animal-Based Systems

Animal-based model systems of bone associated or cellular proliferative or differentiative disease may include, but are not limited to, non-recombinant and engineered transgenic animals.

Non-recombinant animal models for bone associated or cellular proliferative or differentiative disease may include, for example, genetic models.

Additionally, animal models exhibiting bone associated or cellular proliferative or differentiative disease symptoms may be engineered by using, for example, 17906 gene sequences described above, in conjunction with techniques for producing transgenic animals that are well known to those of skill in the art. For example, 17906 gene sequences may be introduced into, and overexpressed in, the genome of the animal of interest, or, if endogenous 17906 gene sequences are present, they may either be overexpressed or, alternatively, be disrupted in order to underexpress or inactivate 17906 gene expression.

The host cells of the invention can also be used to produce non-human transgenic animals. For example, in one embodiment, a host cell of the invention is a fertilized oocyte or an embryonic stem cell into which 17906-coding sequences have been introduced. Such host cells can then be used to create non-human transgenic animals in which exogenous 17906 sequences have been introduced into their genome or homologous recombinant animals in which endogenous 17906 sequences have been altered. Such animals are useful for studying the function and/or activity of a 17906 and for identifying and/or evaluating modulators of 17906 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 17906 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a 17906 encoding nucleic acid into the male pronuclei of a fertilized oocyte, e.g., by microinjection, retroviral infection, and allowing the oocyte to develop in a pseudopregnant female foster animal. The 17906 cDNA sequence of SEQ ID NO:1 can be introduced as a transgene into the genome of a non-human animal. Alternatively, a nonhuman homologue of a human 17906 gene, such as a mouse or rat 17906 gene, can be used as a transgene. Alternatively, a 17906 gene homologue, such as another 17906 family member, can be isolated based on hybridization to the 17906 cDNA sequences of SEQ ID NO:1 and used as a transgene. Intronic sequences and polyadenylation signals can also be included in the transgene to increase the efficiency of expression of the transgene. A tissue-specific regulatory sequence(s) can be operably linked to a 17906 transgene to direct expression of a 17906 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 17906 transgene in its genome and/or expression of 17906 mRNA in tissues or cells of the animals. A transgenic founder animal can then be used to breed additional animals carrying the transgene. Moreover, transgenic animals carrying a transgene encoding a 17906 protein can further be bred to other transgenic animals carrying other transgenes.

To create a homologous recombinant animal, a vector is prepared which contains at least a portion of a 17906 gene into which a deletion, addition or substitution has been introduced to thereby alter, e.g., functionally disrupt, the 17906 gene. The 17906 gene can be a human gene (e.g., the cDNA of SEQ ID NO:1), but more preferably, is a nonhuman homologue of a human 17906 gene (e.g., a cDNA isolated by stringent hybridization with the nucleotide sequence of SEQ ID NO:1). For example, a mouse 17906 gene can be used to construct a homologous recombination nucleic acid molecule, e.g., a vector, suitable for altering an endogenous 17906 gene in the mouse genome. In a preferred embodiment, the homologous recombination nucleic acid molecule is designed such that, upon homologous recombination, the endogenous 17906 gene is functionally disrupted (i.e., no longer encodes a functional protein; also referred to as a “knock out” vector). Alternatively, the homologous recombination nucleic acid molecule can be designed such that, upon homologous recombination, the endogenous 17906 gene is mutated or otherwise altered but still encodes functional protein (e.g., the upstream regulatory region can be altered to thereby alter the expression of the endogenous 17906 protein). In the homologous recombination nucleic acid molecule, the altered portion of the 17906 gene is flanked at its 5′ and 3′ ends by additional nucleic acid sequence of the 17906 gene to allow for homologous recombination to occur between the exogenous 17906 gene carried by the homologous recombination nucleic acid molecule and an endogenous 17906 gene in a cell, e.g., an embryonic stem cell. The additional flanking 17906 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 17906 gene has homologously recombined with the endogenous 17906 gene are selected (see e.g., Li, E. et al. (1992) Cell 69:915). The selected cells can then injected into a blastocyst of an animal (e.g. a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 17906 transgenic animals that express 17906 mRNA or a 17906 peptide (detected immunocytochemically, using antibodies directed against 17906 epitopes) at easily detectable levels should then be further evaluated to identify those animals which display characteristic bone associated or cellular proliferative or differentiative disease symptoms. Such symptoms may include, for example, increased prevalence and size of fatty streaks and/or bone associated or cellular proliferative or differentiative disease plaques.

Additionally, specific cell types within the transgenic animals may be analyzed and assayed for cellular phenotypes characteristic of bone associated or cellular proliferative or differentiative disease. In the case of monocytes, such phenotypes may include but are not limited to increases in rates of LDL uptake, adhesion to bone, breast, lung, colon or ovarian cells, transmigration, foam cell formation, fatty streak formation, and production of foam cell specific products. Cellular phenotypes may include a particular cell type's pattern of expression of genes associated with bone associated or cellular proliferative or differentiative disease as compared to known expression profiles of the particular cell type in animals exhibiting bone associated or cellular proliferative or differentiative disease symptoms.

An alternative animal-based model system of bone associated or cellular proliferative or differentiative disease useful in the present invention is found in ovariectomized rats as described by Dunstan et al. (Dunstan, C. R. et al. J. Bone Miner Res. Vol. 14(6):953-9, 1999). After ovariectomy (OVX), adult female rats begin losing bone density, which can lead to conditions similar to severe osteoporosis. As such the ovariectomized rats may be examined for the prevention of bone density decreases or for new bone formation after various treatments, including those of the present invention.

Ovariectomized rats may also be used as a model for orally administered agents to assay for effects on bone loss, as shown by Mundy et al. (Mundy, G. et al. Science, Vol. 386:1946-1949, 1999). Mundy et al. also describe another animal-based model system of detecting bone growth by injection into the subcutaneous tissue overlying the murine calvaria in mice (Mundy, G. et al. Science, Vol. 386:1946, 1999). Lastly, Mundy et al. describe a model system based on neonatal murine calvarial (skullcap) bones in organ culture as well as an in vitro model for bone formation based on a murine osteoblast cell line. Both of these may be used as described below for cell-based model systems.

B. Cell-Based Systems

Cells that contain and express 17906 gene sequences which encode a 17906 protein, and/or exhibit cellular phenotypes associated with bone associated or cellular proliferative or differentiative disease, may be used to identify compounds that exhibit anti-bone associated or cellular proliferative or differentiative disease activity. Such cells may include non-recombinant monocyte cell lines, such as U937 (ATCC# CRL-1593), THP-1 (ATCC#TIB-202), and P388D1 (ATCC# TIB-63); hepatic cells such as human Hepa; as well as generic mammalian cell lines such as HeLa cells and COS cells, e.g., COS-7 (ATCC# CRL-1651). Further, such cells may include recombinant, transgenic cell lines. For example, the bone associated or cellular proliferative or differentiative disease animal models of the invention, discussed above, may be used to generate cell lines, containing one or more cell types involved in bone associated or cellular proliferative or differentiative disease, that can be used as cell culture models for this disorder. While primary cultures derived from the bone associated or cellular proliferative or differentiative disease transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in bone associated or cellular proliferative or differentiative disease may be transfected with sequences capable of increasing or decreasing the amount of 17906 gene expression within the cell. For example, 17906 gene sequences may be introduced into, and overexpressed in, the genome of the cell of interest, or, if endogenous 17906 gene sequences are present, they may be either overexpressed or, alternatively disrupted in order to underexpress or inactivate 17906 gene expression.

In order to overexpress a 17906 gene, the coding portion of the 17906 gene may be ligated to a regulatory sequence which is capable of driving gene expression in the cell type of interest, e.g., a bone, breast, lung, colon or ovarian cell. Such regulatory regions will be well known to those of skill in the art, and may be utilized in the absence of undue experimentation. Recombinant methods for expressing target genes are described above.

For underexpression of an endogenous 17906 gene sequence, such a sequence may be isolated and engineered such that when reintroduced into the genome of the cell type of interest, the endogenous 17906 alleles will be inactivated. Preferably, the engineered 17906 sequence is introduced via gene targeting such that the endogenous 17906 sequence is disrupted upon integration of the engineered 17906 sequence into the cell's genome. Transfection of host cells with 17906 genes is discussed, above.

Cells treated with compounds or transfected with 17906 genes can be examined for phenotypes associated with bone associated or cellular proliferative or differentiative disease. In the case of osteocytes, such phenotypes include but are not limited to expression of cytokines or growth factors. Expression of cytokines or growth factors may be measured using any of the assays described herein.

Similarly, bone, breast, lung, colon or ovarian cells can be treated with test compounds or transfected with genetically engineered 17906 genes. The bone, breast, lung, colon or ovarian cells can then be examined for phenotypes associated with bone associated or cellular proliferative or differentiative disease, including, but not limited to changes in cellular morphology, cell proliferation, and cell migration; or for the effects on production of other proteins involved in bone associated or cellular proliferative or differentiative disease such as adhesion molecules (e.g., ICAM, VCAM), PDGF, and Eselectin.

Transfection of 17906 nucleic acid may be accomplished by using standard techniques (described in, for example, Ausubel (1989) supra). Transfected cells should be evaluated for the presence of the recombinant 17906 gene sequences, for expression and accumulation of 17906 mRNA, and for the presence of recombinant 17906 protein production. In instances wherein a decrease in 17906 gene expression is desired, standard techniques may be used to demonstrate whether a decrease in endogenous 17906 gene expression and/or in 17906 protein production is achieved.

8. Pharmaceutical Compositions

Active compounds for use in the methods of the invention can be incorporated into pharmaceutical compositions suitable for administration. As used herein, the language “active compounds” includes 17906 nucleic acid molecules, fragments of 17906 proteins, and anti-17906 antibodies, as well as identified compounds that modulate 17906 gene expression, synthesis, and/or activity. Such compositions typically comprise the compound, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a 17906 protein or a 17906 ligand) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. In one embodiment, a therapeutically effective dose refers to that amount of an active compound sufficient to result in amelioration of symptoms of bone associated or cellular proliferative or differentiative disease.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (ie., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g. a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, β-interferon, #-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

9. Isolated Nucleic Acid Molecules

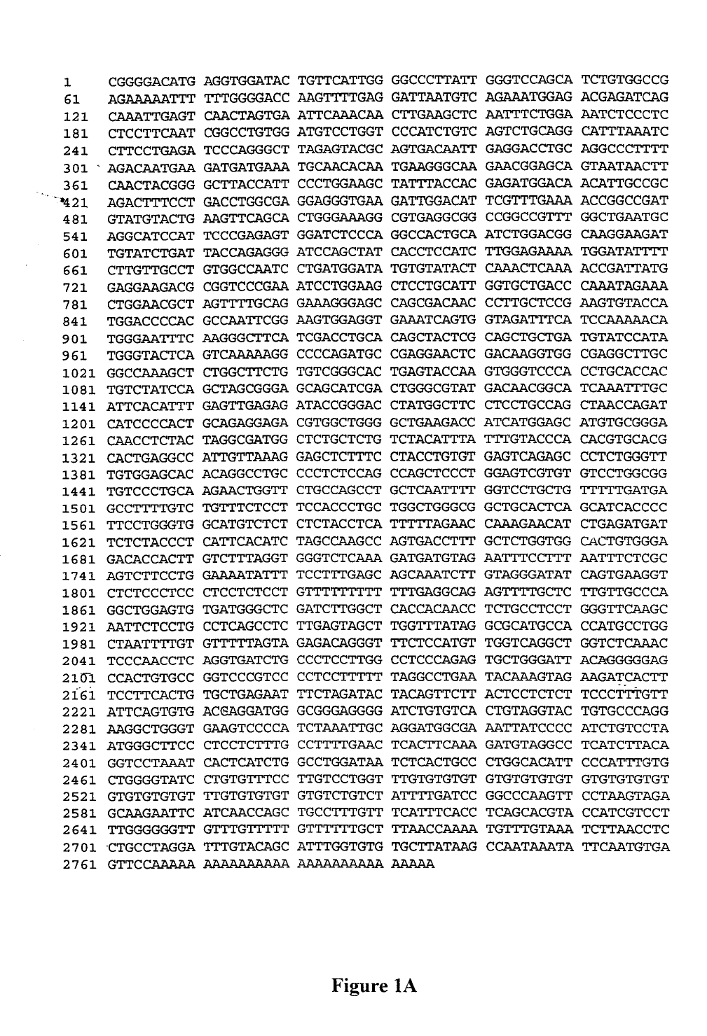

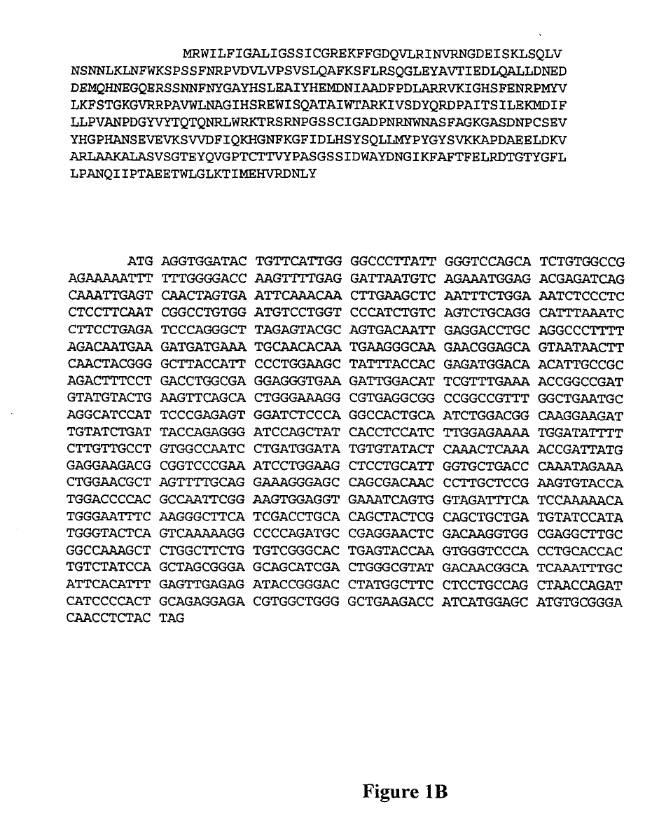

The nucleotide sequence of the isolated human 17906 cDNA and the predicted amino acid sequence of the human 17906 polypeptide are shown in FIG. 1 and in SEQ ID NOs:1 and 2, respectively. The nucleotide sequence encoding human 17906 is identical to the nucleic acid molecule with GenBank Accession Number AF095719 (Huang, H. et al. Cancer Res. (1999) 59(12):2981-2988).

The human 17906 gene, which is approximately 2795 nucleotides in length, encodes a protein having a molecular weight of approximately 46.4 kD and which is approximately 422 amino acid residues in length.

The methods of the invention include the use of isolated nucleic acid molecules that encode 17906 proteins or biologically active portions thereof, as well as nucleic acid fragments sufficient for use as hybridization probes to identify 17906-encoding nucleic acid molecules (e.g., 17906 mRNA) and fragments for use as PCR primers for the amplification or mutation of 17906 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated 17906 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1, as a hybridization probe, 17906 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to 17906 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to the human 17906 cDNA. This cDNA comprises sequences encoding the human 17906 protein (i.e., “the coding region of SEQ ID NO:1”).

In another preferred embodiment, an isolated nucleic acid molecule of the invention comprises a nucleic acid molecule which is a complement of the nucleotide sequence shown in SEQ ID NO:1, or a portion of any of this nucleotide sequence. A nucleic acid molecule which is complementary to the nucleotide sequence shown in SEQ ID NO:1 is one which is sufficiently complementary to the nucleotide sequence shown in SEQ ID NO:1 such that it can hybridize to the nucleotide sequence shown in SEQ ID NO:1, thereby forming a stable duplex.

In still another preferred embodiment, an isolated nucleic acid molecule of the present invention comprises a nucleotide sequence which is at least about 30%, 35%, 40%, 45%, 50%, 55%, 60%, 65%, 70%, 75%, 80%, 85%, 90%, 95%, 98% or more identical to the entire length of the nucleotide sequence shown in SEQ ID NO:1, or a portion of any of this nucleotide sequence.

Moreover, the nucleic acid molecule of the invention can comprise only a portion of the nucleic acid sequence of SEQ ID NO:1, for example, a fragment which can be used as a probe or primer or a fragment encoding a portion of a 17906 protein, e.g., a biologically active portion of a 17906 protein. The nucleotide sequence determined from the cloning of the 17906 gene allows for the generation of probes and primers designed for use in identifying and/or cloning other 17906 family members, as well as 17906 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, of an anti-sense sequence of SEQ ID NO:1, or of a naturally occurring allelic variant or mutant of SEQ ID NO:1. In one embodiment, a nucleic acid molecule of the present invention comprises a nucleotide sequence which is greater than 100, 100-200, 200-300, 300-400, 400-500, 500-600, 600-700, 700-800, or more nucleotides in length and hybridizes under stringent hybridization conditions to a nucleic acid molecule of SEQ ID NO:1.

Probes based on the 17906 nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 17906 protein, such as by measuring a level of a 17906-encoding nucleic acid in a sample of cells from a subject e.g., detecting 17906 mRNA levels or determining whether a genomic 17906 gene has been mutated or deleted.

A nucleic acid fragment encoding a “biologically active portion of a 17906 protein” can be prepared by isolating a portion of the nucleotide sequence of SEQ ID NO:1 which encodes a polypeptide having a 17906 biological activity (the biological activities of the 17906 protein is described herein), expressing the encoded portion of the 17906 protein (e.g., by recombinant expression in vitro) and assessing the activity of the encoded portion of the 17906 protein.

The methods of the invention further encompass nucleic acid molecules that differ from the nucleotide sequence shown in SEQ ID NO:1, due to degeneracy of the genetic code and thus encode the same 17906 protein as those encoded by the nucleotide sequence shown in SEQ ID NO:1. In another embodiment, an isolated nucleic acid molecule of the invention has a nucleotide sequence encoding a protein having an amino acid sequence shown in SEQ ID NO:2.

In addition to the 17906 nucleotide sequence shown in SEQ ID NO:1, it will be appreciated by those skilled in the art that DNA sequence polymorphisms that lead to changes in the amino acid sequences of the 17906 protein may exist within a population (e.g., the human population). Such genetic polymorphism in the 17906 gene may exist among individuals within a population due to natural allelic variation. As used herein, the terms “gene” and “recombinant gene” refer to nucleic acid molecules which include an open reading frame encoding a 17906 protein, preferably a mammalian 17906 protein, and can further include non-coding regulatory sequences, and introns.

Allelic variants of human 17906 include both functional and non-functional 17906 proteins. Functional allelic variants are naturally occurring amino acid sequence variants of the human 17906 protein that maintain the ability to bind a 17906 ligand or substrate and/or modulate cell proliferation and/or migration mechanisms. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human 17906 protein that do not have the ability to either bind a 17906 ligand or substrate and/or modulate cell proliferation and/or migration mechanisms. Non-functional allelic variants will typically contain a non-conservative substitution, a deletion, or insertion or premature truncation of the amino acid sequence of SEQ ID NO:2, or a substitution, insertion or deletion in critical residues or critical regions.

The methods of the present invention may further use non-human orthologues of the human 17906 protein. Orthologues of the human 17906 protein are proteins that are isolated from non-human organisms and possess the same 17906 ligand binding and/or modulation of cell proliferation and/or migration mechanisms of the human 17906 protein. Orthologues of the human 17906 protein can readily be identified as comprising an amino acid sequence that is substantially identical to SEQ ID NO:2.

Moreover, nucleic acid molecules encoding other 17906 family members and, thus, which have a nucleotide sequence which differs from the 17906 sequence of SEQ ID NO:1 are intended to be within the scope of the invention. For example, another 17906 cDNA can be identified based on the nucleotide sequence of human 17906. Moreover, nucleic acid molecules encoding 17906 proteins from different species, and which, thus, have a nucleotide sequence which differs from the 17906 sequence of SEQ ID NO:1 are intended to be within the scope of the invention. For example, a mouse 17906 cDNA can be identified based on the nucleotide sequence of human 17906.

Nucleic acid molecules corresponding to natural allelic variants and homologues of the 17906 cDNA of the invention can be isolated based on their homology to the 17906 nucleic acid disclosed herein using the cDNAs disclosed herein, or a portion thereof, as a hybridization probe according to standard hybridization techniques under stringent hybridization conditions. Nucleic acid molecules corresponding to natural allelic variants and homologues of the 17906 cDNA of the invention can further be isolated by mapping to the same chromosome or locus as the 17906 gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1. In other embodiment, the nucleic acid is at least 30, 50, 100, 150, 200, 250, 300, 350, 400, 450, 500, 550, 600, 650, 700, 750, 800, 1000, 1200, or more nucleotides in length. As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2 × SSC, 0.1% SDS at 50° C., preferably at 55° C., more preferably at 60° C., and even more preferably at 65° C. Ranges intermediate to the above-recited values, e.g., at 60-65° C. or at 55-60° C. are also intended to be encompassed by the present invention. Preferably, an isolated nucleic acid molecule of the invention that hybridizes under stringent conditions to the sequence of SEQ ID NO:1 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the 17906 sequences that may exist in the population, the skilled artisan will further appreciate that changes can be introduced by mutation into the nucleotide sequence of SEQ ID NO:1, thereby leading to changes in the amino acid sequence of the encoded 17906 protein, without altering the functional ability of the 17906 protein. For example, nucleotide substitutions leading to amino acid substitutions at “non-essential” amino acid residues can be made in the sequence of SEQ ID NO:1. A “non-essential” amino acid residue is a residue that can be altered from the wild-type sequence of 17906 (e.g., the sequence of SEQ ID NO:2) without altering the biological activity, whereas an “essential” amino acid residue is required for biological activity. For example, amino acid residues that are conserved among the 17906 proteins of the present invention are predicted to be particularly unamenable to alteration. Furthermore, additional amino acid residues that are conserved between the 17906 proteins of the present invention and other members of the G protein-coupled receptor family are not likely to be amenable to alteration.

Accordingly, the methods of the invention may include the use of nucleic acid molecules encoding 17906 proteins that contain changes in amino acid residues that are not essential for activity. Such 17906 proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 30%, 35%, 40%, 45%, 50%, 55%, 60%, 65%, 70%, 75%, 80%, 85%, 90%, 95%, 98% or more identical to SEQ ID NO:2.

An isolated nucleic acid molecule encoding a 17906 protein identical to the protein of SEQ ID NO:2 can be created by introducing one or more nucleotide substitutions, additions or deletions into the nucleotide sequence of SEQ ID NO:1 such that one or more amino acid substitutions, additions or deletions are introduced into the encoded protein. Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 17906 protein is preferably replaced with another amino acid residue from the same side chain family. Alternatively, in another embodiment, mutations can be introduced randomly along all or part of a 17906 coding sequence, such as by saturation mutagenesis, and the resultant mutants can be screened for 17906 biological activity to identify mutants that retain activity. Following mutagenesis of SEQ ID NO:1, the encoded protein can be expressed recombinantly and the activity of the protein can be determined.

In a preferred embodiment, a mutant 17906 protein can be assayed for the ability to (1) interact with a non-17906 protein molecule, e.g., a 17906 ligand or substrate; (2) activate a 17906-dependent signal transduction pathway; or (3) modulate cell proliferation and/or migration mechanisms, or modulate the expression of cell surface adhesion molecules.

In addition to the nucleic acid molecules encoding 17906 proteins described herein, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 17906 coding strand, or to only a portion thereof. In one embodiment, an antisense nucleic acid molecule is antisense to a “coding region” of the coding strand of a nucleotide sequence encoding 17906. The term “coding region” refers to the region of the nucleotide sequence comprising codons which are translated into amino acid residues (e.g., the coding region of human 17906 corresponds to nucleotides 8-1273 of SEQ ID NO:1). In another embodiment, the antisense nucleic acid molecule is antisense to a “noncoding region” of the coding strand of a nucleotide sequence encoding 17906. The term “noncoding region” refers to 5′ and 3′ sequences which flank the coding region that are not translated into amino acids (i.e., also referred to as 5′ and 3′ untranslated regions).

Given the coding strand sequences encoding 17906 disclosed herein (e.g., nucleotides 8-1273 of SEQ ID NO:1), antisense nucleic acids of the invention can be designed according to the rules of Watson and Crick base pairing. The antisense nucleic acid molecule can be complementary to the entire coding region of 17906 mRNA, but more preferably is an oligonucleotide which is antisense to only a portion of the coding or noncoding region of 17906 mRNA. For example, the antisense oligonucleotide can be complementary to the region surrounding the translation start site of 17906 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In yet another embodiment, the 17906 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of 17906 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 17906 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of 17906 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 17906 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridizationtriggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

10. Isolated 17906 Proteins and Anti-17906 Antibodies

The methods of the invention include the use of isolated 17906 proteins, and biologically active portions thereof, as well as polypeptide fragments suitable for use as immunogens to raise anti-17906 antibodies.

Isolated proteins of the present invention, preferably 17906 proteins, have an amino acid sequence sufficiently identical to the amino acid sequence of SEQ ID NO:2, or are encoded by a nucleotide sequence sufficiently identical to SEQ ID NO:1. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, a “17906 activity”, “biological activity of 17906” or “functional activity of 17906”, refers to an activity exerted by a 17906 protein, polypeptide or nucleic acid molecule on a 17906 responsive cell (e.g., a bone, breast, lung, colon or ovarian cell) or tissue, or on a 17906 protein substrate, as determined in vivo, or in vitro, according to standard techniques. In one embodiment, a 17906 activity is a direct activity, such as an association with a 17906 target molecule. As used herein, a “target molecule” or “binding partner” is a molecule with which a 17906 protein binds or interacts in nature, such that 17906-mediated function is achieved. A 17906 target molecule can be a non-17906 molecule or a 17906 protein or polypeptide of the present invention. In an exemplary embodiment, a 17906 target molecule is a 17906 ligand. Alternatively, a 17906 activity is an indirect activity, such as a cellular signaling activity mediated by interaction of the 17906 protein with a 17906 ligand. Preferably, a 17906 activity is the ability to act as a signal transduction molecule and to modulate bone, breast, lung, colon or ovarian cell proliferation, differentiation, and/or migration. Accordingly, another embodiment of the invention features isolated 17906 proteins and polypeptides having a 17906 activity.

In one embodiment, native 17906 proteins can be isolated from cells or tissue sources by an appropriate purification scheme using standard protein purification techniques. In another embodiment, 17906 proteins are produced by recombinant DNA techniques. Alternative to recombinant expression, a 17906 protein or polypeptide can be synthesized chemically using standard peptide synthesis techniques.

An “isolated” or “purified” protein or biologically active portion thereof is substantially free of cellular material or other contaminating proteins from the cell or tissue source from which the 17906 protein is derived, or substantially free from chemical precursors or other chemicals when chemically synthesized. The language “substantially free of cellular material” includes preparations of 17906 protein in which the protein is separated from cellular components of the cells from which it is isolated or recombinantly produced. In one embodiment, the language “substantially free of cellular material” includes preparations of 17906 protein having less than about 30% (by dry weight) of non-17906 protein (also referred to herein as a “contaminating protein”), more preferably less than about 20% of non-17906 protein, still more preferably less than about 10% of non-17906 protein, and most preferably less than about 5% non-17906 protein. When the 17906 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of 17906 protein in which the protein is separated from chemical precursors or other chemicals which are involved in the synthesis of the protein. In one embodiment, the language “substantially free of chemical precursors or other chemicals” includes preparations of 17906 protein having less than about 30% (by dry weight) of chemical precursors or non-17906 chemicals, more preferably less than about 20% chemical precursors or non-17906 chemicals, still more preferably less than about 10% chemical precursors or non-17906 chemicals, and most preferably less than about 5% chemical precursors or non-17906 chemicals.

As used herein, a “biologically active portion” of a 17906 protein includes a fragment of a 17906 protein which participates in an interaction between a 17906 molecule and a non-17906 molecule. Biologically active portions of a 17906 protein include peptides comprising amino acid sequences sufficiently identical to or derived from the amino acid sequence of the 17906 protein, e.g., the amino acid sequence shown in SEQ ID NO:2, which include less amino acids than the full length 17906 protein, and exhibit at least one activity of a 17906 protein. Typically, biologically active portions comprise a domain or motif with at least one activity of the 17906 protein, e.g., modulating cell proliferation mechanisms. A biologically active portion of a 17906 protein can be a polypeptide which is, for example, 10, 25, 50, 100, 200, or more amino acids in length. Biologically active portions of a 17906 protein can be used as targets for developing agents which modulate a 17906 mediated activity, e.g., a cell proliferation mechanism. A biologically active portion of a 17906 protein comprises a protein in which regions of the protein are deleted, can be prepared by recombinant techniques and evaluated for one or more of the functional activities of a native 17906 protein.

In a preferred embodiment, the 17906 protein has an amino acid sequence shown in SEQ ID NO:2. In other embodiments, the 17906 protein is substantially identical to SEQ ID NO:2, and retains the functional activity of the protein of SEQ ID NO:2, yet differs in amino acid sequence due to natural allelic variation or mutagenesis, as described in detail in subsection I above. Accordingly, in another embodiment, the 17906 protein is a protein which comprises an amino acid sequence at least about 30%, 35%, 40%, 45%, 50%, 55%, 60%, 65%, 70%, 75%, 80%, 85%, 90%, 95%, 98% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 17906 amino acid sequence of SEQ ID NO:2 having 516 amino acid residues, at least 136, preferably at least 181, more preferably at least 227, even more preferably at least 272, and even more preferably at least 317, 362 or 408 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Myers and Miller, Comput. Appl. Biosci. 4:1117 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score 100, wordlength=12 to obtain nucleotide sequences homologous to 17906 nucleic acid molecules of the invention. BLAST protein searches can be performed with the XBLAST program, score=100, wordlength=3 to obtain amino acid sequences homologous to 17906 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The methods of the invention may also use 17906 chimeric or fusion proteins. As used herein, a 17906 “chimeric protein” or “fusion protein” comprises a 17906 polypeptide operatively linked to a non-17906 polypeptide. A “17906 polypeptide” refers to a polypeptide having an amino acid sequence corresponding to 17906, whereas a “non-17906 polypeptide” refers to a polypeptide having an amino acid sequence corresponding to a protein which is not substantially homologous to the 17906 protein, e.g., a protein which is different from the 17906 protein and which is derived from the same or a different organism. Within a 17906 fusion protein the 17906 polypeptide can correspond to all or a portion of a 17906 protein. In a preferred embodiment, a 17906 fusion protein comprises at least one biologically active portion of a 17906 protein. In another preferred embodiment, a 17906 fusion protein comprises at least two biologically active portions of a 17906 protein. Within the fusion protein, the term “operatively linked” is intended to indicate that the 17906 polypeptide and the non-17906 polypeptide are fused in-frame to each other. The non-17906 polypeptide can be fused to the Nterminus or C-terminus of the 17906 polypeptide.

For example, in one embodiment, the fusion protein is a GST-17906 fusion protein in which the 17906 sequences are fused to the C-terminus of the GST sequences. Such fusion proteins can facilitate the purification of recombinant 17906. In another embodiment, the fusion protein is a 17906 protein containing a heterologous signal sequence at its N-terminus. In certain host cells (e.g., mammalian host cells), expression and/or secretion of 17906 can be increased through use of a heterologous signal sequence.

The 17906 fusion proteins of the invention can be incorporated into pharmaceutical compositions and administered to a subject in vivo. The 17906 fusion proteins can be used to affect the bioavailability of a 17906 ligand. Use of 17906 fusion proteins may be useful therapeutically for the treatment of disorders caused by, for example, (i) aberrant modification or mutation of a gene encoding a 17906 protein; (ii) mis-regulation of the 17906 gene; and (iii) aberrant post-translational modification of a 17906 protein. In one embodiment, a 17906 fusion protein may be used to treat a bone associated or cellular proliferative or differentiative disorder. In another embodiment, a 17906 fusion protein may be used to treat a bone, breast, lung, ovary or colon cell disorder.

Moreover, the 17906-fusion proteins of the invention can be used as immunogens to produce anti-17906 antibodies in a subject, to purify 17906 ligands and in screening assays to identify molecules which inhibit the interaction of 17906 with a 17906 substrate.

Preferably, a 17906 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 17906-encoding nucleic acid can be cloned into such an expression vector such that the fusion moiety is linked in-frame to the 17906 protein.

The methods of the present invention may also include the use of variants of the 17906 protein which function as either 17906 agonists (mimetics) or as 17906 antagonists. Variants of the 17906 protein can be generated by mutagenesis, e.g., discrete point mutation or truncation of a 17906 protein. An agonist of the 17906 protein can retain substantially the same, or a subset, of the biological activities of the naturally occurring form of a 17906 protein. An antagonist of a 17906 protein can inhibit one or more of the activities of the naturally occurring form of the 17906 protein by, for example, competitively modulating a 17906-mediated activity of a 17906 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 17906 protein.

In one embodiment, variants of a 17906 protein which function as either 17906 agonists (mimetics) or as 17906 antagonists can be identified by screening combinatorial libraries of mutants, e.g., truncation mutants, of a 17906 protein for 17906 protein agonist or antagonist activity. In one embodiment, a variegated library of 17906 variants is generated by combinatorial mutagenesis at the nucleic acid level and is encoded by a variegated gene library. A variegated library of 17906 variants can be produced by, for example, enzymatically ligating a mixture of synthetic oligonucleotides into gene sequences such that a degenerate set of potential 17906 sequences is expressible as individual polypeptides, or alternatively, as a set of larger fusion proteins (e.g., for phage display) containing the set of 17906 sequences therein. There are a variety of methods which can be used to produce libraries of potential 17906 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 17906 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a 17906 protein coding sequence can be used to generate a variegated population of 17906 fragments for screening and subsequent selection of variants of a 17906 protein. In one embodiment, a library of coding sequence fragments can be generated by treating a double stranded PCR fragment of a 17906 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 17906 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 17906 proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 17906 variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated 17906 library. For example, a library of expression vectors can be transfected into a cell line, e.g., a bone, breast, lung, ovary or colon cell line, which ordinarily responds to a 17906 ligand in a particular 17906-dependent manner. The transfected cells are then contacted with a 17906 ligand and the effect of expression of the mutant on signaling by the 17906 receptor can be detected, e.g., by monitoring the generation of an intracellular second messenger (e.g., calcium, cAMP, IP₃, or diacylglycerol), the phosphorylation profile of intracellular proteins, cell proliferation and/or migration, the expression profile of cell surface adhesion molecules, or the activity of a 17906-regulated transcription factor. Plasmid DNA can then be recovered from the cells which score for inhibition, or alternatively, potentiation of signaling by the 17906 receptor, and the individual clones further characterized.

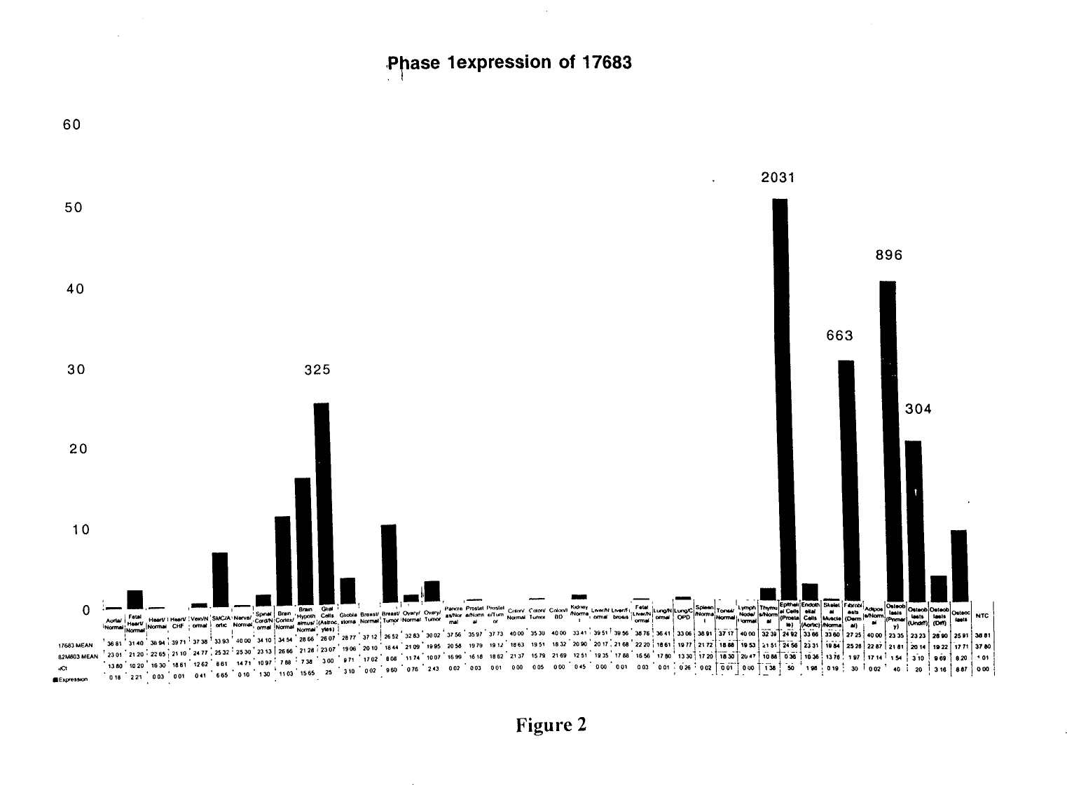

An isolated 17906 protein, or a portion or fragment thereof, can be used as an immunogen to generate antibodies that bind 17906 using standard techniques for polyclonal and monoclonal antibody preparation. A full-length 17906 protein can be used or, alternatively, the invention provides antigenic peptide fragments of 17906 for use as immunogens. The antigenic peptide of 17906 comprises at least 8 amino acid residues of the amino acid sequence shown in SEQ ID NO:2 and encompasses an epitope of 17906 such that an antibody raised against the peptide forms a specific immune complex with 17906. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of 17906 that are located on the surface of the protein, e.g., hydrophilic regions, as well as regions with high antigenicity (see FIG. 2).

A 17906 immunogen typically is used to prepare antibodies by immunizing a suitable subject, (e.g., rabbit, goat, mouse or other mammal) with the immunogen. An appropriate immunogenic preparation can contain, for example, recombinantly expressed 17906 protein or a chemically synthesized 17906 polypeptide. The preparation can further include an adjuvant, such as Freund's complete or incomplete adjuvant, or similar immunostimulatory agent. Immunization of a suitable subject with an immunogenic 17906 preparation induces a polyclonal anti-17906 antibody response.

Accordingly, another aspect of the invention pertains to anti-17906 antibodies. The term “antibody” as used herein refers to immunoglobulin molecules and immunologically active portions of immunoglobulin molecules, i.e., molecules that contain an antigen binding site which specifically binds (immunoreacts with) an antigen, such as 17906. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 17906. The term “monoclonal antibody” or “monoclonal antibody composition”, as used herein, refers to a population of antibody molecules that contain only one species of an antigen binding site capable of immunoreacting with a particular epitope of 17906. A monoclonal antibody composition thus typically displays a single binding affinity for a particular 17906 protein with which it immunoreacts.

Polyclonal anti-17906 antibodies can be prepared as described above by immunizing a suitable subject with a 17906 immunogen. The anti-17906 antibody titer in the immunized subject can be monitored over time by standard techniques, such as with an enzyme linked immunosorbent assay (ELISA) using immobilized 17906. If desired, the antibody molecules directed against 17906 can be isolated from the mammal (e.g., from the blood) and further purified by well known techniques, such as protein A chromatography to obtain the IgG fraction. At an appropriate time after immunization, e.g., when the anti-17906 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem .255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBVhybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, New York (1980); E. A. Lemer (1981) Yale J. Biol. Med., 54:387-402; M. L. Gefter et al. (1977) Somatic Cell Genet. 3:231-36). Briefly, an immortal cell line (typically a mycloma) is fused to lymphocytes (typically splenocytes) from a mammal immunized with a 17906 immunogen as described above, and the culture supernatants of the resulting hybridoma cells are screened to identify a hybridoma producing a monoclonal antibody that binds 17906.

Any of the many well known protocols used for fusing lymphocytes and immortalized cell lines can be applied for the purpose of generating an anti-17906 monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g. the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 17906, e.g., using a standard ELISA assay.

Alternative to preparing monoclonal antibody-secreting hybridomas, a monoclonal anti-17906 antibody can be identified and isolated by screening a recombinant combinatorial immunoglobulin library (e.g., an antibody phage display library) with 17906 to thereby isolate immunoglobulin library members that bind 17906. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et a! PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBOJ 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-17906 antibodies, such as chimeric and humanized monoclonal antibodies, comprising both human and non-human portions, which can be made using standard recombinant DNA techniques, can also be used in the methods of the present invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. CancerInst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-17906 antibody (e.g., monoclonal antibody) can be used to isolate 17906 by standard techniques, such as affinity chromatography or immunoprecipitation. An anti-17906 antibody can facilitate the purification of natural 17906 from cells and of recombinantly produced 17906 expressed in host cells. Moreover, an anti-17906 antibody can be used to detect 17906 protein (e.g., in a cellular lysate or cell supernatant) in order to evaluate the abundance and pattern of expression of the 17906 protein. Anti17906 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include

¹²⁵I, ¹³¹I, ³⁵S or ³H.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification and Characterization of Human 17906 cDNAs

The human 17906 sequence (FIG. 1A; SEQ ID NO:1), which is approximately 2795 nucleotides long including untranslated regions, contains a predicted methionine-initiated coding sequence (SEQ ID NO:3; FIG. 1B) of about 1266 nucleotides (nucleotides 8 to 1273 of SEQ ID NO:1). The coding sequence encodes a 422 amino acid protein (SEQ ID NO:2; FIG. 1B).

### Example 2

### Expression and Tissue Distribution of 17906 mRNA

Northern blot hybridizations with various RNA samples can be performed under standard conditions and washed under stringent conditions, i.e., 0.2× SSC at 65° C. A DNA probe corresponding to all or a portion of the 17906 cDNA (SEQ ID NOs:1 or 3) can be used. The DNA is radioactively labeled with 32P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

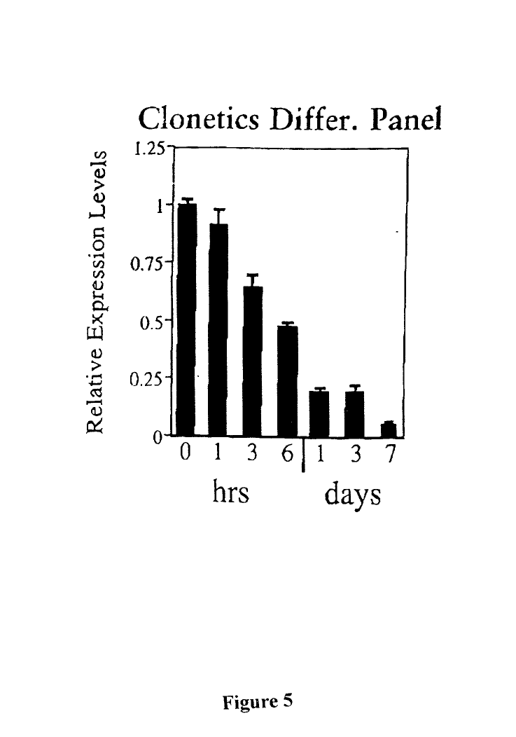

TaqMan real-time quantitative RT-PCR was used to detect the presence of RNA transcript corresponding to human 17906 in several tissues. It was found that the corresponding orthologs of 17906 are expressed in a variety of tissues. The results of this screening are shown in FIGS. 2-9.

Reverse Transcriptase PCR (RT-PCR) was used to detect the presence of RNA transcript corresponding to human 17906 in RNA prepared from cells and tissues related to osteoblasts. Expression of 17906 was assessed in several tissues. A relatively low expression of the transcript was found in differentiated osteoblasts, and relatively high expression of the transcript was found in primary cultured osteoblasts.

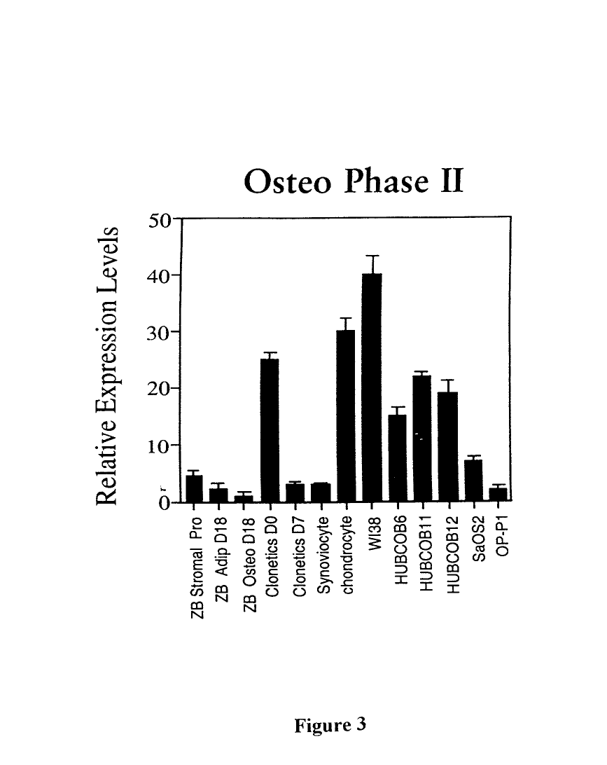

Relative expression levels of the 17906 was assessed in osteogenic cells and adipogenic cells using TaqMan PCR. The results of this comparison are shown in FIG. 3.

FIGS. 2 and 3 also depict results of TaqMan PCR used to assess the expression of 17906 in several cellular models of osteoporosis.

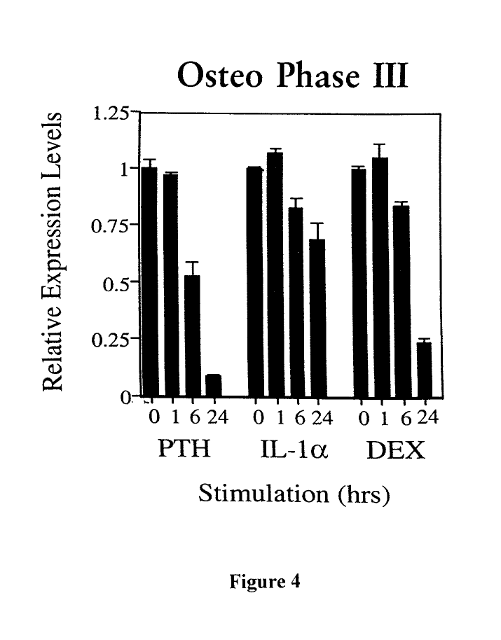

Relative mRNA expression levels of the 17906 gene was also assessed in osteoblasts stimulated with parathyroid hormone (PTH), interleukin-1α (IL-1α), and dexamethasone (DEX) as shown in FIG. 4.

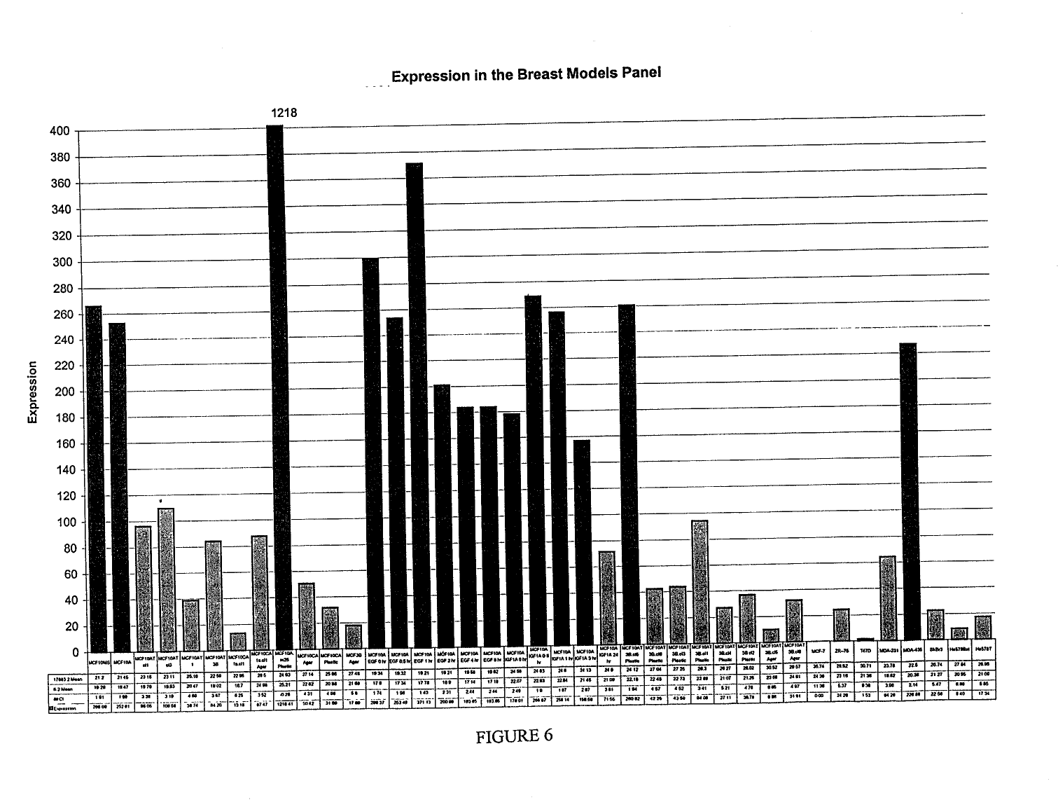

Reverse Transcriptase PCR (RT-PCR) was used to detect the presence of RNA transcript corresponding to human 17906 in RNA prepared from tumor and normal tissues. FIGS. 6-9 illustrate the relative expression levels and tissue distribution of the 17906 genes in various tissues using Taq Man PCR. If a subject has a disease characterized by underexpression or overexpression of a 17906 gene, modulators which have a stimulatory or inhibitory effect on carboxypeptidase activity (e.g., carboxypeptidase gene expression) can be administered to individuals to treat (prophylactically or therapeutically) carboxypeptidase-associated disorders.

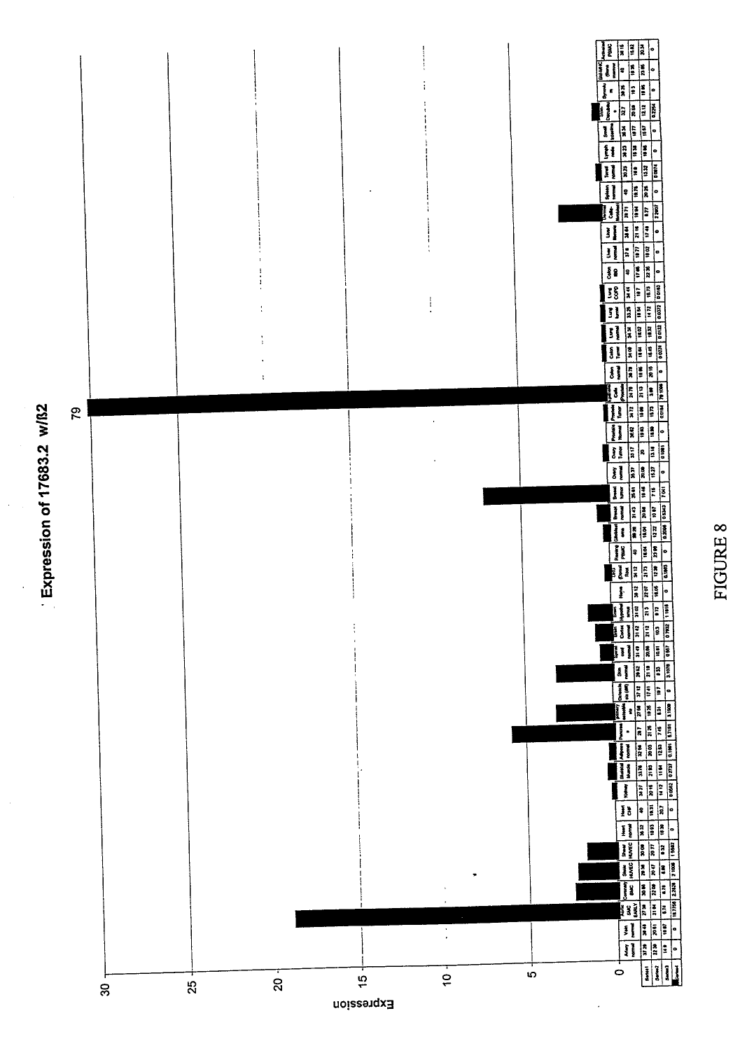

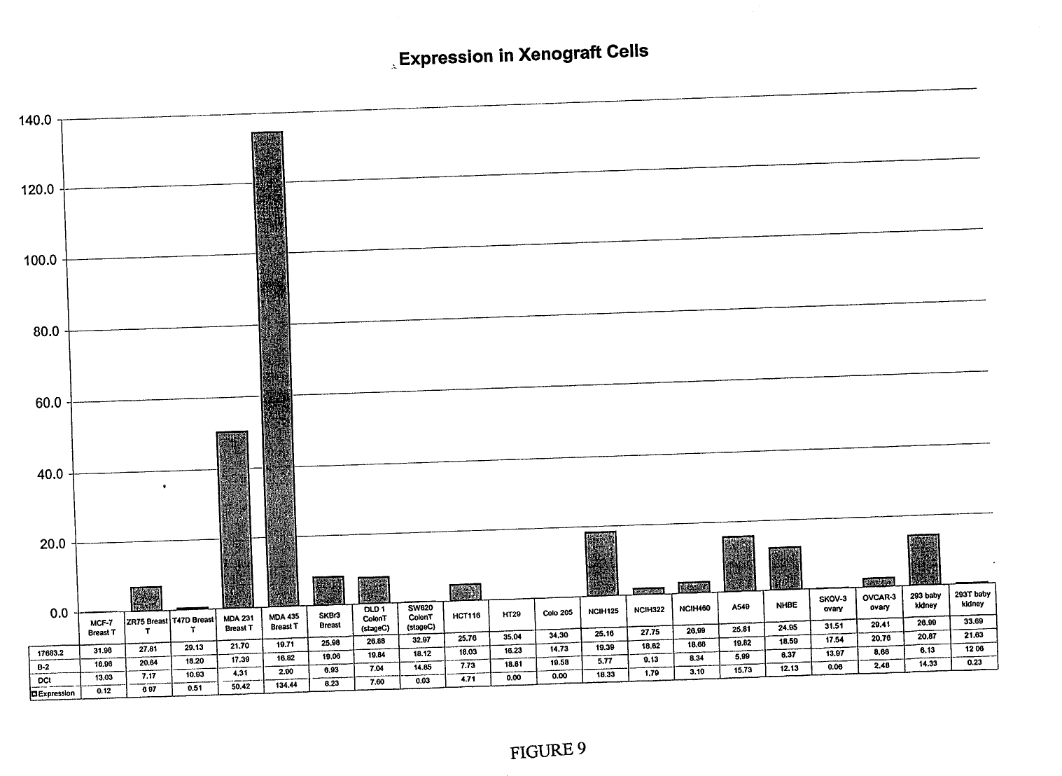

FIG. 8 illustrates the relative expression levels of 17906 in various tissues using TaqMan PCR, and significant expression in prostate epithelial cells. Variable expression was found in xenographs of cell lines tested as shown in FIG. 9 for 17906; the highest expression for 17906 was found in MDA-435 breast tumor cell line.

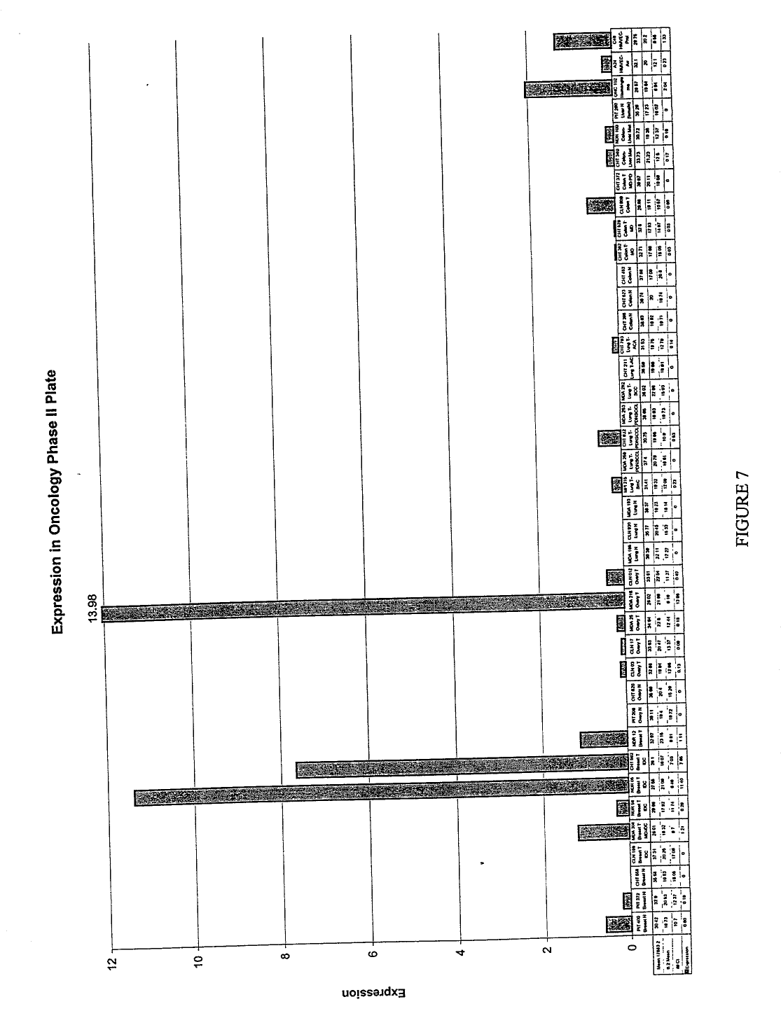

FIG. 6 shows significant expression in the MCF10A (m25 Plastic) breast model. FIG. 7 shows some 17906 expression of 17906 mRNA relative to a no template control showing an increased expression in 4/6 breast tumors in comparison to normal breast tissues, 5/5 ovarian tumors in comparison to normal ovarian tissues, 3/7 lung tumors in comparison to normal lung tissues, and 3/4 colon tumors and 2/2 colon metastases in comparison to normal colon tissues. Again, expression was detected using Taq Man analysis.

As seen by these results, 17906 molecules have been found to be overexpressed in some tumors or cells, where the molecules may be inappropriately propagating either cell proliferation or cell survival signals or have aberrant protein kinase activity. As such, 17906 molecules may serve as specific and novel identifiers of such tumor cells or disorders.

Further, modulators of the 17906 molecules are useful for the treatment of cancer. For example, inhibitors of the 17906 molecules are useful for the treatment of cancer where 17906 is upregulated in tumor cells such as ovarian, colon, breast, and lung cancer and colon metastases, and are useful as a diagnostic.

### Example 3

### Expression of Recombinant 17906 Protein in Bacterial Cells

In this example, 17906 is expressed as a recombinant glutathione-S-transferase (GST) fusion polypeptide in E. coli and the fusion polypeptide is isolated and characterized. Specifically, 17906 is fused to GST and this fusion polypeptide is expressed in E. coli, e.g., strain PEB199. Expression of the GST-17906, fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant 17906 Protein in COS Cells

To express the 17906 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 17906 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 17906 DNA sequence is amplified by PCR using two primers. The 5′ primer contains the restriction site of interest followed by approximately twenty nucleotides of the 17906 coding sequence starting from the initiation codon; the 3′ end sequence contains complementary sequences to the other restriction site of interest, a translation stop codon, the HA tag or FLAG tag and the last 20 nucleotides of the 17906 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 17906 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 17906-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 17906 polypeptide is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with 35S-methionine (or 35S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 17906 coding sequence is cloned directly into the polylinker of the pcDNA/Amp vector using the appropriate restriction sites. The resulting plasmid is transfected into COS cells in the manner described above, and the expression of the 17906 polypeptide is detected by radiolabelling and immunoprecipitation using a 17906 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated 17906 nucleic acid molecule selected from the group consisting of:
a) a nucleic acid molecule comprising a nucleotide sequence which is at least 60% identical to the nucleotide sequence of SEQ ID NO:1, SEQ ID NO:3, or the nucleotide sequence of the DNA insert of the plasmid deposited with ATCC as Accession Number ______;
b) a nucleic acid molecule comprising a fragment of at least 15 nucleotides of the nucleotide sequence of SEQ ID NO:1, SEQ ID NO:3, or the nucleotide sequence of the DNA insert of the plasmid deposited with ATCC as Accession Number ______;
c) a nucleic acid molecule which encodes a polypeptide comprising the amino acid sequence of SEQ ID NO:2, or the amino acid sequence encoded by the cDNA insert of the plasmid deposited with the ATCC as Accession Number ______;
d) a nucleic acid molecule which encodes a fragment of a polypeptide comprising the amino acid sequence of SEQ ID NO:2, or the amino acid sequence encoded by the cDNA insert of the plasmid deposited with the ATCC as Accession Number ______, wherein the fragment comprises at least 15 contiguous amino acids of SEQ ID NO:2, or the amino acid sequence encoded by the cDNA insert of the plasmid deposited with the ATCC as Accession Number ______;
e) a nucleic acid molecule which encodes a naturally occurring allelic variant of a polypeptide comprising the amino acid sequence of SEQ ID NO:2, or the amino acid sequence encoded by the cDNA insert of the plasmid deposited with the ATCC as Accession Number ______, wherein the nucleic acid molecule hybridizes to a nucleic acid molecule comprising SEQ ID NO:1, SEQ ID NO:3, or a complement thereof, under stringent conditions;
f) a nucleic acid molecule comprising the nucleotide sequence of SEQ ID NO:1, SEQ ID NO:3, or the nucleotide sequence of the DNA insert of the plasmid deposited with ATCC as Accession Number ______; and
g) a nucleic acid molecule which encodes a polypeptide comprising the amino acid sequence of SEQ ID NO:2, or the amino acid sequence encoded by the cDNA insert of the plasmid deposited with the ATCC as Accession Number ______.

2. The isolated nucleic acid molecule of claim 1, which is the nucleotide sequence SEQ ID NO:1.

3. An isolated 17906 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, SEQ ID NO:3, or the nucleotide sequence of the DNA insert of the plasmid deposited with ATCC as Accession Number ______, or a complement thereof;
b) a naturally occurring allelic variant of a polypeptide comprising the amino acid sequence of SEQ ID NO:2, or the amino acid sequence encoded by the cDNA insert of the plasmid deposited with the ATCC as Accession Number ______, wherein the polypeptide is encoded by a nucleic acid molecule which hybridizes to a nucleic acid molecule comprising SEQ ID NO:1, SEQ ID NO:3, or a complement thereof under stringent conditions;
c) a fragment of a polypeptide comprising the amino acid sequence of SEQ ID NO:2, or the amino acid sequence encoded by the cDNA insert of the plasmid deposited with the ATCC as Accession Number ______, wherein the fragment comprises at least 15 contiguous amino acids of SEQ ID NO:2; and
d) the amino acid sequence of SEQ ID NO:2.

4. A method of identifying a nucleic acid molecule associated with a bone disorder, cancer or a cellular proliferation and/or differentiation disorder comprising:
a) contacting a sample from a subject with or at risk of developing a bone disorder, cancer or a cellular proliferation and/or differentiation disorder comprising nucleic acid molecules with a hybridization probe comprising at least 25 contiguous nucleotides of SEQ ID NO:1 as defined in claim 2; and
b) detecting the presence of a nucleic acid molecule in the sample that hybridizes to the probe, thereby identifying a nucleic acid molecule associated with a bone disorder, cancer or a cellular proliferation and/or differentiation disorder.

5. A method of identifying a nucleic acid associated with a bone disorder, cancer or a cellular proliferation and/or differentiation disorder comprising:
a) contacting a sample from a subject having a bone disorder, cancer or a cellular proliferation and/or differentiation disorder or at risk of developing a bone disorder, cancer or a cellular proliferation and/or differentiation disorder comprising nucleic acid molecules with a first and a second amplification primer, the first primer comprising at least 25 contiguous nucleotides of SEQ ID NO:1 as defined in claim 2 and the second primer comprising at least 25 contiguous nucleotides from the complement of SEQ ID NO:1;
b) incubating the sample under conditions that allow nucleic acid amplification; and
c) detecting the presence of a nucleic acid molecule in the sample that is amplified, thereby identifying the nucleic acid molecule associated with a bone disorder, cancer or a cellular proliferation and/or differentiation disorder.

6. A method of identifying a polypeptide associated with a bone disorder, cancer or a cellular proliferation and/or differentiation disorder comprising:
a) contacting a sample comprising polypeptides with a 17906 binding partner of the 17906 polypeptide defined in claim 3; and
b) detecting the presence of a polypeptide in the sample that binds to the 17906 binding partner, thereby identifying the polypeptide associated with a bone disorder, cancer or a cellular proliferation and/or differentiation disorder.

7. A method of identifying a subject having a bone disorder, cancer or a cellular proliferation and/or differentiation disorder or at risk for developing a bone disorder, cancer or a cellular proliferation and/or differentiation disorder comprising:
a) contacting a sample obtained from the subject comprising nucleic acid molecules with a hybridization probe comprising at least 25 contiguous nucleotides of SEQ ID NO:1 as defined in claim 2; and
b) detecting the presence of a nucleic acid molecule in the sample that hybridizes to the probe, thereby identifying a subject having a bone disorder, cancer or a cellular proliferation and/or differentiation disorder or at risk for developing a bone disorder, cancer or a cellular proliferation and/or differentiation disorder.

8. A method of identifying a subject having a bone disorder, cancer or a cellular proliferation and/or differentiation disorder or at risk for developing a bone disorder, cancer or a cellular proliferation and/or differentiation disorder comprising:
a) contacting a sample obtained from the subject comprising nucleic acid molecules with a first and a second amplification primer, the first primer comprising at least 25 contiguous nucleotides of SEQ ID NO:1 as defined in claim 2 and the second primer comprising at least 25 contiguous nucleotides from the complement of SEQ ID NO:1;
b) incubating the sample under conditions that allow nucleic acid amplification; and
c) detecting the presence of a nucleic acid molecule in the sample that is amplified, thereby identifying a subject having a bone disorder, cancer or a cellular proliferation and/or differentiation disorder or at risk for developing a bone disorder, cancer or a cellular proliferation and/or differentiation disorder.

9. A method of identifying a subject having a bone disorder, cancer or a cellular proliferation and/or differentiation disorder or at risk for developing a bone disorder, cancer or a cellular proliferation and/or differentiation disorder comprising:
a) contacting a sample obtained from the subject comprising polypeptides with a 17906 binding partner of the 17906 polypeptide defined in claim 3; and
b) detecting the presence of a polypeptide in the sample that binds to the 17906 binding partner, thereby identifying a subject having a bone disorder, cancer or a cellular proliferation and/or differentiation disorder or at risk for developing a bone disorder, cancer or a cellular proliferation and/or differentiation disorder.

10. A method for identifying a compound capable of treating a bone disorder, cancer or a cellular proliferation and/or differentiation disorder characterized by aberrant 17906 nucleic acid expression or 17906 polypeptide activity comprising assaying the ability of the compound to modulate 17906 nucleic acid expression or 17906 polypeptide activity, thereby identifying a compound capable of treating a bone disorder, cancer or a cellular proliferation and/or differentiation disorder characterized by aberrant 17906 nucleic acid expression or 17906 polypeptide activity.

11. A method for treating a subject having a bone disorder, cancer or a cellular proliferation and/or differentiation disorder or at risk of developing a bone disorder, cancer or a cellular proliferation and/or differentiation disorder comprising administering to the subject a 17906 modulator of the nucleic acid molecule defined in claim 1 or the polypeptide encoded by the nucleic acid molecule or contacting a cell with a 17906 modulator.

12. The method defined in claim 11 wherein said cancer is selected from the group consisting of ovarian, lung, colon, and breast cancer.

13. The method defined in claim 11 wherein the disorder is osteoporosis or relates to osteoblast differentiation.

14. The method of claim 11, wherein the 17906 modulator is
a) a small molecule;
b) peptide;
c) phosphopeptide;
d) anti-17906 antibody;
e) a 17906 polypeptide comprising the amino acid sequence of SEQ ID NO:2, or a fragment thereof;
f) a 17906 polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:2, wherein the polypeptide is encoded by a nucleic acid molecule which hybridizes to a complement of a nucleic acid molecule consisting of SEQ ID NO:1 at 6× SSC at 45° C., followed by one or more washes in 0.2× SSC, 0.1% SDS at 65° C.

15. The method of claim 11, wherein the 17906 modulator is
a) an antisense 17906 nucleic acid molecule;
b) is a ribozyme;
c) the nucleotide sequence of SEQ ID NO:1, or a fragment thereof;
d) a nucleic acid molecule encoding a polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, wherein the nucleic acid molecule which hybridizes to a complement of a nucleic acid molecule consisting of SEQ ID NO:1 at 6× SSC at 45° C., followed by one or more washes in 0.2× SSC, 0.1% SDS at 65° C.; or
f) a gene therapy vector.

16. A method for evaluating the efficacy of a treatment of a bone disorder, cancer or a cellular proliferation and/or differentiation disorder, in a subject, comprising:
treating a subject with a protocol under evaluation;
assessing the expression level of a 17906 nucleic acid molecule defined in claim 1 or 17906 polypeptide encoded by the 17906 nucleic acid molecule,
wherein a change in the expression level of 17906 nucleic acid or 17906 polypeptide after the treatment, relative to the level before the treatment, is indicative of the efficacy of the treatment of a bone disorder, cancer or a cellular proliferation and/or differentiation disorder.

17. A method of diagnosing a bone disorder, cancer or a cellular proliferation and/or differentiation disorder in a subject, comprising:
evaluating the expression or activity of a 17906 nucleic acid molecule defined in claim 1 or a 17906 polypeptide encoded by the 17906 nucleic acid molecule, such that a difference in the level of 17906 nucleic acid or 17906 polypeptide relative to a normal subject or a cohort of normal subjects is indicative of a bone disorder, cancer or a cellular proliferation and/or differentiation disorder.
